(19) United States
(12) Patent Application Publication
(10) Pub. No.: US 2020/0223071 A1
(43) Pub. Date: Jul. 16, 2020

(54) EXOSUIT SYSTEMS AND METHODS WITH ACCESSORY DEVICE SUPPORT

(71) Applicant: SEISMIC HOLDINGS, INC., Menlo Park, CA (US)

(72) Inventors: Richard M. Mahoney, Los Altos, CA (US); Esther Leong, Cave Creek, AZ (US); Mallory L. Tayson-Frederick, San Francisco, CA (US)

(21) Appl. No.: 16/739,607

(22) Filed: Jan. 10, 2020

Related U.S. Application Data
(60) Provisional application No. 62/790,907, filed on Jan. 10, 2019.

(51) Int. Cl. B25J 13/08; B25J 9/00; A61B 5/11; A63B 21/00; A61F 5/01; G05B 19/409

(52) U.S. Cl. CPC B25J 13/081; B25J 9/0006; A61B 5/1116; A61F 2005/0188; A61F 5/0102; G05B 19/409; G05B 2219/40305; A63B 21/00181

(57) ABSTRACT

Exosuit systems can operate in conjunction with an accessory device or other remote device that is designed to control and provide information related to the exosuit system. The accessory device can be used by a user to engage in manual control or symbiosis control of the exosuit system. The accessory device can display graphical elements such as buttons that succinctly convey operational status of various exosuit components and assistance operations.

### CROSS-REFERENCE TO RELATED APPLICATIONS

This application claims priority to U.S. Provisional Patent Application No. 62/790,907, filed Jan. 10, 2019, the disclosure of which is incorporated by reference in its entirety.

### BACKGROUND

Wearable robotic systems have been developed for augmentation of humans' natural capabilities, or to replace functionality lost due to injury or illness.

### SUMMARY

Exosuit systems can operate in conjunction with an accessory device or other remote device that is designed to control and provide information related to the exosuit system. The accessory device can be used by a user to engage in manual control or symbiosis control of the exosuit system. The accessory device can display graphical elements such as buttons that succinctly convey operational status of various exosuit components and assistance operations.

In one embodiment, a device for use with an exosuit is provided that includes a housing; communications circuitry operative to communicate with the exosuit; an interactive display exposed through an opening of the housing; and a processor positioned within the housing and is configured to: display, on the interactive display, a plurality of exosuit control buttons, wherein each exosuit control button corresponds to at least one power layer of the exosuit responsible for implementing exosuit assistance associated with that particular exosuit control button; receive data from the exosuit via the communications circuitry, the data comprising data corresponding to the at least one power layer associated with each of the exosuit control buttons; and change a visual element of at least one of the exosuit control buttons based on the received data.

In one embodiment, the visual element comprises a countdown timer that graphically illustrates when the at least one power layer associated with one of exosuit control buttons is going to activate.

In one embodiment, when the countdown timer expires, the at least one power layer associated with one of the exosuit control buttons is activated.

In one embodiment, the visual element changes from an inactive state to an active state, wherein in the inactive state, the at least one power layer associated with one of the exosuit control buttons is not activated, and wherein in the active state, the at least one power layer associated with one of the exosuit control buttons is activated.

In one embodiment, the processor is further configured to: receive user selection of one of the displayed plurality of exosuit control buttons; and display, on the interactive display, a configuration screen including an ON/OFF toggle switch operative to enable or disable the at least one power layer associated with the selected exosuit control button; and a force quantity control to set a level of assistance force applied by the at least one power layer associated with the selected exosuit control button.

In one embodiment, the processor is further configured to receive a user swipe input in a first direction; and display, on the interactive display, battery status of at least one of the power layers in response to the user swipe input in the first direction, wherein the battery status of each of the power layers is received from the exosuit via the communications circuitry.

In one embodiment, the processor is further configured to receive a user swipe input in a second direction and display, on the interactive display, a settings screen including a posture calibration button that, when selected, is operative to calibrate the exosuit, and an exosuit pairing button that, when selected, is operative to enable a user to pair the device with the exosuit.

In one embodiment, the processor is further configured to display, on the interactive display, a symbiosis button; receive user selection of the symbiosis button; display, on the interactive display, in response to user selection of the symbiosis button a scrollable list including a plurality of exosuit assistance movement buttons each associated with an ON/OFF toggle switch; and a plurality of timer buttons corresponding to respective ones of the plurality of exosuit control buttons, wherein each one of the plurality of timer buttons specifies a time limit and is further selectable to enable a user to define the time limit.

In one embodiment, a method for operating an accessory device that is in communication with an exosuit system is provided. The method includes initiating a symbiosis mode of control for the exosuit in response to user selection of a symbiosis button on the accessory device. The symbiosis mode of control includes receiving data from the exosuit system, the data comprising a determined body posture of the user wearing the exosuit suit; displaying, on an interactive display of the accessory device, a first exosuit control button comprising a first timer; activating the first timer in response to receipt of the determined body posture, wherein activating the first timer comprises displaying a countdown of the first timer and during the countdown of the first timer, the exosuit system is configuring at least one flexible linear actuator associated with at least one power layer to actuate a base tone commensurate with the determined body posture; and when the first timer has elapsed, changing a display element of the first exosuit control button to indicate that exosuit enabled assistance support is ready for activation, and wherein when exosuit enabled assistance support is triggered to go active, the exosuit system transitions from the base tone to active assistance support.

In one embodiment, the symbiosis mode of control further includes displaying, on the interactive display, a cancel button; receiving user selection of the cancel button, wherein selection of the cancel button ends the active assistance support; and changing the display element to indicate the exosuit enabled assistance support is not active in response to user selection of the cancel button.

In one embodiment, the symbiosis mode of control further including receiving a new determined body posture while the first timer is counting down; resetting the first timer in response to receipt of the new determined body posture; and re-activating the first timer after the first timer has been reset and displaying the countdown of the first timer.

In one embodiment, the determined body posture is a physiological determination of a positional state or predicted motion state of a user of the exosuit system.

In one embodiment, the base tone comprises pre-emptive tensioning of the at least one flexible linear actuator that is used to provide the exosuit enabled assistance support.

In one embodiment, a time duration of the first timer is an amount of time configurable by a user via manipulation of a setting in the interactive display.

In one embodiment, the time duration is the amount of time the user remained in the determined posture in order to transition to the base tone.

In one embodiment, the symbiosis mode of control further includes receiving a trigger to transition from the base tone to the active assistance support; and operating the at least one power layer to provide the active assistance support.

In one embodiment, the trigger is an elapse of the first timer.

In one embodiment, the trigger is included in the received data provided by the exosuit system or is a input received via the interactive display.

In one embodiment, a method for operating an accessory device that is in communication with an exosuit system is provided. The method includes displaying, on an interactive display, a home page comprising a symbiosis/manual toggle switch and a plurality of exosuit control buttons, wherein each of the exosuit control buttons corresponds to an exosuit assistance operation; receiving user selection of the symbiosis/manual toggle switch to operate the exosuit system in a manual mode, wherein the manual mode requires the user to select one of the exosuit control buttons to activate the exosuit assistance operation corresponding to the selected exosuit control button; and receiving user selection of the symbiosis/manual toggle switch to operate the exosuit system in a symbiosis mode, wherein the symbiosis mode automatically activates an exosuit assistance operation based on data received from the exosuit system, and wherein the exosuit control button corresponding to the exosuit assistance operation is displayed with at least one display element that changes depending on a status of the exosuit control button.

In one embodiment, the at least one display element comprises a countdown timer that indicates when the exosuit assistance operation is ready for activation, the method further including displaying a configuration screen corresponding to a selected one of the plurality of exosuit control buttons, the configuration screen comprising a force quantity control to set a level of assistance force applied by the exosuit suit during activation of the exosuit assistance operation corresponding to the selected exosuit control button.

In one embodiment, the at least one display element indicates whether the exosuit assistance operation is active or not active.

In one embodiment, the method further includes displaying a configuration screen corresponding to a selected one of the plurality of exosuit control buttons, the configuration screen comprising a force quantity control to set a level of assistance force applied by the exosuit suit during activation of the exosuit assistance operation corresponding to the selected exosuit control button.

In one embodiment, the method further includes displaying a status screen comprising information related to the operation of the exosuit system.

In one embodiment, the information comprises battery information, exosuit component failure notification, exosuit connectivity notification, and any combination thereof.

In one embodiment, the at least one display element comprises a countdown timer that indicates when the exosuit assistance operation is ready for activation.

### BRIEF DESCRIPTION OF THE DRAWINGS

Various objects, features, and advantages of the disclosed subject matter can be more fully appreciated with reference to the following detailed description of the disclosed subject matter when considered in connection with the following drawings, in which like reference numerals identify like elements.

FIGS. 1A, 1B, and 1C show front, back, and side views of a base layer of an exosuit according to an embodiment.

FIGS. 1D, 1E, and 1F show front, back, and side views, respectively, of a power layer according to an embodiment.

FIGS. 1G and 1H show respective front and back views of a human male's musculature anatomy, according to an embodiment.

FIGS. 1I and 1J show front and side views of an illustrative exosuit having several power layer segments that approximate many of the muscles shown in FIGS. 1G and 1H, according to various embodiments.

FIGS. 2A and 2B show front and back view of an illustrative exosuit according to an embodiment.

FIG. 3 shows an illustrative symbiosis exosuit system according to an embodiment.

FIG. 4 shows illustrative an process for implementing the symbiosis exosuit system according to an embodiment.

FIG. 5 illustrates an exemplary personal electronic device that may be used in connection with an exosuit according to an embodiment.

FIG. 6 illustrates a block diagram of some of the components of a device according to some embodiments.

FIG. 7 shows an illustrative process for implementing timer based symbiosis of an exosuit according to an embodiment.

FIG. 8 shows an illustrative process for implemented timer free symbiosis modes according to an embodiment.

FIGS. 9A, 9B, 9C, 9D, 9E, 9F, and 9G shows several different personal device screens according to various embodiments.

FIGS. 10A, 10B, 10C, and 10D show different screens that may be shown as part of lumbar support via timer according to an embodiment.

FIGS. 11A, 11B, and 11C show different screens that may be shown as part of manually activated lumbar support according to an embodiment.

FIGS. 12A and 12B show different screens showing symbiosis mode being turned OFF according to an embodiment.

FIGS. 13A, 13B, 13C, and 13D show different screens related to sit-to-stand according to an embodiment.

FIGS. 14A, 14B, 14C, and 14D show different screens that show standing support and lumbar support via timers according to an embodiment.

FIGS. 15A, 15B, 15C, and 15D show different screens related to lumbar support according to an embodiment.

FIGS. 16A, 16B, 16C, and 16D show different screens related to standing support according to an embodiment.

FIGS. 17A, 17B, 17C, and 17D show different screens related to adjusting lumbar support according to an embodiment.

FIGS. 18A, 18B, 18C, and 18D show different screens related to adjusting lumbar support according to an embodiment.

FIGS. 19A and 19B show how the timers for lumbar support and standing support can be set according to various embodiments.

FIGS. 20A, 20B, 20C, and 20D show different screens that may be displayed according to various embodiments.

FIGS. 21A, 21B, 21C, 21D, 21E, 21F, and 21G show different calibration screens that may be displayed according to various embodiments.

FIGS. 22A and 22B show different battery status screens that may be displayed according to various embodiments.

FIG. 23 shows a warning screen indicating the personal device lost connection with the exosuit according to an embodiment.

FIGS. 24A, 24B, and 24C show alert screens that may be displayed according to various embodiments.

FIGS. 25A, 25B, and 25C show high heat alert screens that may be displayed according to various embodiments.

FIGS. 26A, 26B, 26C, and 26D show other alert screens that may be displayed according to various embodiments.

FIG. 27 shows an illustrative process according to an embodiment.

FIG. 28 illustrates an example exosuit according to an embodiment.

FIG. 29 is a schematic illustrating elements of a exosuit according to an embodiment.

### DETAILED DESCRIPTION

In the following description, numerous specific details are set forth regarding the systems, methods and media of the disclosed subject matter and the environment in which such systems, methods and media may operate, etc., in order to provide a thorough understanding of the disclosed subject matter. It can be apparent to one skilled in the art, however, that the disclosed subject matter may be practiced without such specific details, and that certain features, which are well known in the art, are not described in detail in order to avoid complication of the disclosed subject matter. In addition, it can be understood that the examples provided below are exemplary, and that it is contemplated that there are other systems, methods and media that are within the scope of the disclosed subject matter.

In the descriptions that follow, an exosuit or assistive exosuit is a suit that is worn by a wearer on the outside of his or her body. It may be worn under the wearer's normal clothing, over their clothing, between layers of clothing, or may be the wearer's primary clothing itself. The exosuit may be supportive and/or assistive, as it physically supports or assists the wearer in performing particular activities, or can provide other functionality such as communication to the wearer through physical expressions to the body, engagement of the environment, or capturing of information from the wearer. In some embodiments, a powered exosuit system can include several subsystems, or layers. In some embodiments, the powered exosuit system can include more or less subsystems or layers. The subsystems or layers can include the base layer, stability layer, power layer, sensor and controls layer, a covering layer, and user interface/user experience (UI/UX) layer.

The base layer provides the interfaces between the exosuit system and the wearer's body. The base layer may be adapted to be worn directly against the wearer's skin, between undergarments and outer layers of clothing, over outer layers of clothing or a combination thereof, or the base layer may be designed to be worn as primary clothing itself. In some embodiments, the base layer can be adapted to be both comfortable and unobtrusive, as well as to comfortably and efficiently transmit loads from the stability layer and power layer to the wearer's body in order to provide the desired assistance. The base layer can typically comprise several different material types to achieve these purposes. Elastic materials may provide compliance to conform to the wearer's body and allow for ranges of movement. The innermost layer is typically adapted to grip the wearer's skin, undergarments or clothing so that the base layer does not slip as loads are applied. Substantially inextensible materials may be used to transfer loads from the stability layer and power layer to the wearer's body. These materials may be substantially inextensible in one axis, yet flexible or extensible in other axes such that the load transmission is along preferred paths. The load transmission paths may be optimized to distribute the loads across regions of the wearer's body to minimize the forces felt by the wearer, while providing efficient load transfer with minimal loss and not causing the base layer to slip. Collectively, this load transmission configuration within the base layer may be referred to as a load distribution member. Load distribution members refer to flexible elements that distribute loads across a region of the wearer's body. Examples of load distribution members can be found in International Publication No. WO 2016/138264, titled “Flexgrip,” the contents of which are incorporated herein by reference.

The load distribution members may incorporate one or more catenary curves to distribute loads across the wearer's body. Multiple load distribution members or catenary curves may be joined with pivot points, such that as loads are applied to the structure, the arrangement of the load distribution members pivots tightens or constricts on the body to increase the gripping strength. Compressive elements such as battens, rods, or stays may be used to transfer loads to different areas of the base layer for comfort or structural purposes. For example, a power layer component may terminate in the middle back due to its size and orientation requirements, however the load distribution members that anchor the power layer component may reside on the lower back. In this case, one or more compressive elements may transfer the load from the power layer component at the middle back to the load distribution member at the lower back.

The load distribution members may be constructed using multiple fabrication and textile application techniques. For example, the load distribution member can be constructed from a layered woven 45°/90° with bonded edge, spandex tooth, organza (poly) woven 45°/90° with bonded edge, organza (cotton/silk) woven 45°/90°, and Tyvek (non-woven). The load distribution member may be constructed using knit and lacing or horse hair and spandex tooth. The load distribution member may be constructed using channels and/or laces.

The base layer may include a flexible underlayer that is constructed to compress against a portion of the wearer's body, either directly to the skin, or to a clothing layer, and also provides a relatively high grip surface for one or more load distribution members to attach thereto. The load distribution members can be coupled to the underlayer to facilitate transmission of shears or other forces from the members, via the flexible underlayer, to skin of a body segment or to clothing worn over the body segment, to maintain the trajectories of the members relative to such a body segment, or to provide some other functionality. Such a flexible underlayer could have a flexibility and/or compliance that differs from that of the member (e.g., that is less than that of the members, at least in a direction along the members), such that the member can transmit forces along their length and evenly distribute shear forces and/or pressures, via the flexible underlayer, to skin of a body segment to which a flexible body harness is mounted.

Further, such a flexible underlayer can be configured to provide additional functionality. The material of the flexible underlayer could include anti-bacterial, anti-fungal, or other agents (e.g., silver nanoparticles) to prevent the growth of microorganisms. The flexible underlayer can be configured to manage the transport of heat and/or moisture (e.g., sweat) from a wearer to improve the comfort and efficiency of activity of the wearer. The flexible underlayer can include straps, seams, hook-and-loop fasteners, clasps, zippers, or other elements configured to maintain a specified relationship between elements of the load distribution members and aspects of a wearer's anatomy. The underlayer can additionally increase the ease with which a wearer can don and/or doff the flexible body harness and/or a system (e.g., a flexible exosuit system) or garment that includes the flexible body harness. The underlayer can additionally be configured to protect the wearer from ballistic weapons, sharp edges, shrapnel, or other environmental hazards (by including, e.g., panels or flexible elements of para-aramid or other high-strength materials).

The base layer can additionally include features such as size adjustments, openings and electro-mechanical integration features to improve ease of use and comfort for the wearer.

Size adjustment features permit the exosuit to be adjusted to the wearer's body. The size adjustments may allow the suit to be tightened or loosened about the length or circumference of the torso or limbs. The adjustments may comprise lacing, the Boa system, webbing, elastic, hook-and-loop or other fasteners. Size adjustment may be accomplished by the load distribution members themselves, as they constrict onto the wearer when loaded. In one example, the torso circumference may be tightened with corset-style lacing, the legs tightened with hook-and-loop in a double-back configuration, and the length and shoulder height adjusted with webbing and tension-lock fasteners such as cam-locks, D-rings or the like. The size adjustment features in the base layer may be actuated by the power layer to dynamically adjust the base layer to the wearer's body in different positions, in order to maintain consistent pressure and comfort for the wearer. For example, the base layer may be required to tighten on the thighs when standing, and loosen when sitting such that the base layer does not excessively constrict the thighs when seated. The dynamic size adjustment may be controlled by the sensor and controls layer, for example by detecting pressures or forces in the base layer and actuating the power layer to consistently attain the desired force or pressure. This feature does not necessarily cause the suit to provide physical assistance, but can create a more comfortable experience for the wearer, or allow the physical assistance elements of the suit to perform better or differently depending on the purpose of the movement assistance.

Opening features in the base layer may be provided to facilitate donning (putting the exosuit on) and doffing (taking the exosuit off) for the wearer. Opening features may comprise zippers, hook-and-loop, snaps, buttons or other textile fasteners. In one example, a front, central zipper provides an opening feature for the torso, while hook-and-loop fasteners provide opening features for the legs and shoulders. In this case, the hook-and-loop fasteners provide both opening and adjustment features. In other examples, the exosuit may simply have large openings, for example around the arms or neck, and elastic panels that allow the suit to be donned and doffed without specific closure mechanisms. A truncated load distribution member may be simply extended to tighten on the wearer's body. Openings may be provided to facilitate toileting so the user can keep the exosuit on, but only have to remove or open a relatively small portion to use the bathroom.

Electro-mechanical integration features attach components of the stability layer, power layer and sensor and controls layer into the base layer for integration into the exosuit. The integration features may be for mechanical, structural, comfort, protective or cosmetic purposes. Structural integration features anchor components of the other layers to the base layer. For the stability and power layers, the structural integration features provide for load-transmission to the base layer and load distribution members, and may accommodate specific degrees of freedom at the attachment point. For example, a snap or rivet anchoring a stability or power layer element may provide both load transmission to the base layer, as well as a pivoting degree of freedom. Stitched, adhesive, or bonded anchors may provide load transmission with or without the pivoting degree of freedom. A sliding anchor, for example along a sleeve or rail, may provide a translational degree of freedom. Anchors may be separable, such as with snaps, buckles, clasps or hooks; or may be inseparable, such as with stitching, adhesives or other bonding. Size adjustment features as described above may allow adjustment and customization of the stability and power layers, for example to adjust the tension of spring or elastic elements in the passive layer, or to adjust the length of actuators in the power layer.

Other integration features such as loops, pockets, and mounting hardware may simply provide attachment to components that do not have significant load transmission requirements, such as batteries, circuit boards, sensors, or cables. In some cases, components may be directly integrated into textile components of the base layer. For example, cables or connectors may include conductive elements that are directly woven, bonded or otherwise integrated into the base layer.

Electromechanical integration features may also protect or cosmetically hide components of the stability, power or sensor and controls layers. Elements of the stability layer (e.g. elastic bands or springs), power layer (e.g. flexible linear actuators or twisted string actuators) or sensor and controls layer (e.g. cables) may travel through sleeves, tubes, or channels integrated into the base layer, which can both conceal and protect these components. The sleeves, tubes, or channels may also permit motion of the component, for example during actuation of a power layer element. The sleeves, channels, or tubes may comprise resistance to collapse, ensuring that the component remains free and uninhibited within.

Enclosures, padding, fabric coverings, or the like may be used to further integrate components of other layers into the base layer for cosmetic, comfort, or protective purposes. For example, components such as motors, batteries, cables, or circuit boards may be housed within an enclosure, fully or partially covered or surrounded in padded material such that the components do not cause discomfort to the wearer, are visually unobtrusive and integrated into the exosuit, and are protected from the environment. Opening and closing features may additionally provide access to these components for service, removal, or replacement.

In some cases—particularly for exosuits configurable for either provisional use or testing—a tether may allow for some electronic and mechanical components to be housed off the suit. In one example, electronics such as circuit boards and batteries may be over-sized, to allow for added configurability or data capture. If the large size of these components makes it undesirable to mount them on the exosuit, they could be located separately from the suit and connected via a physical or wireless tether. Larger, over-powered motors may be attached to the suit via flexible drive linkages that allow actuation of the power layer without requiring large motors to be attached to the suit. Such over-powered configurations allow optimization of exosuit parameters without constraints requiring all components to be attached or integrated into the exosuit.

Electro-mechanical integration features may also include wireless communication. For example, one or more power layer components may be placed at different locations on the exosuit. Rather than utilizing physical electrical connections to the sensors and controls layer, the sensor and controls layer may communicate with the one or more power layer components via wireless communication protocols such as Bluetooth, ZigBee, ultrawide band, or any other suitable communication protocol. This may reduce the electrical interconnections required within the suit. Each of the one or more power layer components may additionally incorporate a local battery such that each power layer component or group of power layer components are independently powered units that do not require direct electrical interconnections to other areas of the exosuit.

The stability layer provides passive mechanical stability and assistance to the wearer. The stability layer comprises one or more passive (non-powered) spring or elastic elements that generate forces or store energy to provide stability or assistance to the wearer. An elastic element can have an un-deformed, least-energy state. Deformation, e.g. elongation, of the elastic element stores energy and generates a force oriented to return the elastic element toward its least-energy state. For example, elastic elements approximating hip flexors and hip extensors may provide stability to the wearer in a standing position. As the wearer deviates from the standing position, the elastic elements are deformed, generating forces that stabilize the wearer and assist maintaining the standing position. In another example, as a wearer moves from a standing to seated posture, energy is stored in one or more elastic elements, generating a restorative force to assist the wearer when moving from the seated to standing position. Similar passive, elastic elements may be adapted to the torso or other areas of the limbs to provide positional stability or assistance moving to a position where the elastic elements are in their least-energy state.

Elastic elements of the stability layer may be integrated to parts of the base layer or be an integral part of the base layer. For example, elastic fabrics containing spandex or similar materials may serve as a combination base/stability layer. Elastic elements may also include discrete components such as springs or segments of elastic material such as silicone or elastic webbing, anchored to the base layer for load transmission at discrete points, as described above.

The stability layer may be adjusted as described above, both to adapt to the wearer's size and individual anatomy, as well as to achieve a desired amount of pre-tension or slack in components of the stability layer in specific positions. For example, some wearers may prefer more pre-tension to provide additional stability in the standing posture, while others may prefer more slack, so that the passive layer does not interfere with other activities such as ambulation.

The stability layer may interface with the power layer to engage, disengage, or adjust the tension or slack in one or more elastic elements. In one example, when the wearer is in a standing position, the power layer may pre-tension one or more elastic elements of the stability layer to a desired amount for maintaining stability in that position. The pre-tension may be further adjusted by the power layer for different positions or activities. In some embodiments, the elastic elements of the stability layer should be able to generate at least 5 lbs force; preferably at least 50 lbs force when elongated.

The power layer can provide active, powered assistance to the wearer, as well as electromechanical clutching to maintain components of the power or stability layers in a desired position or tension. The power layer can include one or more flexible linear actuators (FLA). An FLA is a powered actuator capable of generating a tensile force between two attachment points, over a give stroke length. An FLA is flexible, such that it can follow a contour, for example around a body surface, and therefore the forces at the attachment points are not necessarily aligned. In some embodiments, one or more FLAs can include one or more twisted string actuators. In the descriptions that follow, FLA refers to a flexible linear actuator that exerts a tensile force, contracts or shortens when actuated. The FLA may be used in conjunction with a mechanical clutch that locks the tension force generated by the FLA in place so that the FLA motor does not have to consume power to maintain the desired tension force. In some embodiments, and FLA can include a motor and one or more twisted strings (or other material that shortens in length when twisted by the motor). Examples of such mechanical clutches are discussed below. In some embodiments, FLAs can include one or more twisted string actuators or flexdrives, as described in further detail in U.S. Pat. No. 9,266,233, titled “Exosuit System,” the contents of which are incorporated herein by reference. FLAs may also be used in connection with electrolaminate clutches, which are also described in the U.S. Pat. No. 9,266,233. The electrolaminate clutch (e.g., clutches configured to use electrostatic attraction to generate controllable forces between clutching elements) may provide power savings by locking a tension force without requiring the FLA to maintain the same force.

The powered actuators, or FLAs, are arranged on the base layer, connecting different points on the body, to generate forces for assistance with various activities. The arrangement can often approximate the wearer's muscles, in order to naturally mimic and assist the wearer's own capabilities. For example, one or more FLAs may connect the back of the torso to the back of the legs, thus approximating the wearer's hip extensor muscles. Actuators approximating the hip extensors may assist with activities such as standing from a seated position, sitting from a standing position, walking, or lifting. Similarly, one or more actuators may be arranged approximating other muscle groups, such as the hip flexors, spinal extensors, abdominal muscles or muscles of the arms or legs.

The one or more FLAs approximating a group of muscles are capable of generating at least 10 lbs over at least a ½ inch stroke length within 4 seconds. In some embodiments, one or more FLAs approximating a group of muscles may be capable of generating at least 250 lbs over a 6-inch stroke within ½ second. Multiple FLAs, arranged in series or parallel, may be used to approximate a single group of muscles, with the size, length, power, and strength of the FLAs optimized for the group of muscles and activities for which they are utilized.

The sensor and controls layer captures data from the suit and wearer, utilizes the sensor data and other commands to control the power layer based on the activity being performed, and provides suit and wearer data to the UX/UI layer for control and informational purposes.

Sensors such as encoders or potentiometers may measure the length and rotation of the FLAs, while force sensors measure the forces applied by the FLAs. Inertial measurement units (IMUs) measure and enable computation of kinematic data (positions, velocities and accelerations) of points on the suit and wearer. These data enable inverse dynamics calculations of kinetic information (forces, torques) of the suit and wearer. Electromyographic (EMG) sensors may detect the wearer's muscle activity in specific muscle groups. Electronic control systems (ECSs) on the suit may use parameters measured by the sensor layer to control the power layer. Data from the IMUs may indicate both the activity being performed, as well as the speed and intensity. For example, a pattern of IMU or EMG data may enable the ECS to detect that the wearer is walking at a specific pace. This information then enables the ECS, utilizing the sensor data, to control the power layer in order to provide the appropriate assistance to the wearer. Stretchable sensors may be used as a strain gauge to measure the strain of the elements in the stability layer, and thereby predict the forces in the elastic elements of the stability layer. Stretchable sensors may be embedded in the base layer or grip layer and used to measure the motion of the fabrics in the base layer and the motion of the body.

Data from the sensor layer may be further provided to the UX/UI layer, for feedback and information to the wearer, caregivers or service providers.

The UX/UI layer comprises the wearer's and others' interaction and experience with the exosuit system. This layer includes controls of the suit itself such as initiation of activities, as well as feedback to the wearer and caregivers. A retail or service experience may include steps of fitting, calibration, training and maintenance of the exosuit system. Other UX/UI features may include additional lifestyle features such as electronic security, identity protection and health status monitoring.

The assistive exosuit can have a user interface for the wearer to instruct the suit which activity is to be performed, as well as the timing of the activity. In one example, a user may manually instruct the exosuit to enter an activity mode via one or more buttons, a keypad, or a tethered device such as a mobile phone. In another example, the exosuit may detect initiation of an activity from the sensor and controls layer, as described previously. In yet another example, the user may speak a desired activity mode to the suit, which can interpret the spoken request to set the desired mode. The suit may be pre-programmed to perform the activity for a specific duration, until another command is received from the wearer, or until the suit detects that the wearer has ceased the activity. The suit may include cease activity features that, when activated, cause the suit to cease all activity. The cease activity features can take into account the motion being performed, and can disengage in a way that takes into account the user's position and motion, and safely returns the user to an unloaded state in a safe posture.

The exosuit may have a UX/UI controller that is defined as a node on another user device, such as a computer or mobile smart phone. The exosuit may also be the base for other accessories. For example, the exosuit may include a cell phone chip so that the suit may be capable of receiving both data and voice commands directly similar to a cell phone, and can communicate information and voice signals through such a node. The exosuit control architecture can be configured to allow for other devices to be added as accessories to the exosuit. For example, a video screen may be connected to the exosuit to show images that are related to the use of the suit. The exosuit may be used to interact with smart household devices such as door locks or can be used to turn on smart televisions and adjust channels and other settings. In these modes, the physical assist of the suit can be used to augment or create physical or haptic experiences for the wearer that are related to communication with these devices. For instance, an email could have a pat on the back as a form of physical emoji that when inserted in the email causes the suit to physically tap the wearer or perform some other type of physical expression to the user that adds emphasis to the written email.

The exosuit may provide visual, audio, or haptic feedback or cues to inform the user of various exosuit operations. For example, the exosuit may include vibration motors to provide haptic feedback. As a specific example, two haptic motors may be positioned near the front hip bones to inform the user of suit activity when performing a sit-to-stand assistive movement. In addition, two haptic motors may be positioned near the back hip bones to inform the user of suit activity when performing a stand-to-sit assistive movement. The exosuit may include one or more light emitting diodes (LEDs) to provide visual feedback or cues. For example, LEDS may be placed near the left and/or right shoulders within the peripheral vision of the user. The exosuit may include a speaker or buzzer to provide audio feedback or cues.

In other instances, the interaction of the FLA's with the body through the body harness and otherwise can be used as a form of haptic feedback to the wearer, where changes in the timing of the contraction of the FLA's can indicate certain information to the wearer. For instance, the number or strength of tugs of the FLA on the waist could indicate the amount of battery life remaining or that the suit has entered a ready state for an impending motion.

The control of the exosuit may also be linked to the sensors that are measuring the movement of the wearer, or other sensors, for instance on the suit of another person, or sensors in the environment. The motor commands described herein may all be activated or modified by this sensor information. In this example, the suit can exhibit its own reflexes such that the wearer, through intentional or unintentional motions, cues the motion profile of the suit. When sitting, for further example, the physical movement of leaning forward in the chair, as if to indicate an intention to stand up, can be sensed by the suit IMU's and be used to trigger the sit to stand motion profile. In one embodiment, the exosuit may include sensors (e.g., electroencephalograph (EEG) sensor) that are able to monitor brain activity may be used to detect a user's desire to perform a particular movement. For example, if the user is sitting down, the EEG sensor may sense the user's desire to stand up and cause the exosuit to prime itself to assist the user in a sit-to-stand assistive movement.

The suit may make sounds or provide other feedback, for instance through quick movements of the motors, as information to the user that the suit has received a command or to describe to the user that a particular motion profile can be applied. In the above reflex control example, the suit may provide a high pitch sound and/or a vibration to the wearer to indicate that it is about to start the movement. This information can help the user to be ready for the suit movements, improving performance and safety. Many types of cues are possible for all movements of the suit.

Control of the suit includes the use of machine learning techniques to measure movement performance across many instances of one or of many wearers of suits connected via the internet, where the calculation of the best control motion for optimizing performance and improving safety for any one user is based on the aggregate information in all or a subset of the wearers of the suit. The machine learning techniques can be used to provide user specific customization for exosuit assistive movements. For example, a particular user may have an abnormal gait (e.g., due to a car accident) and thus is unable to take even strides. The machine learning may detect this abnormal gait and compensate accordingly for it.

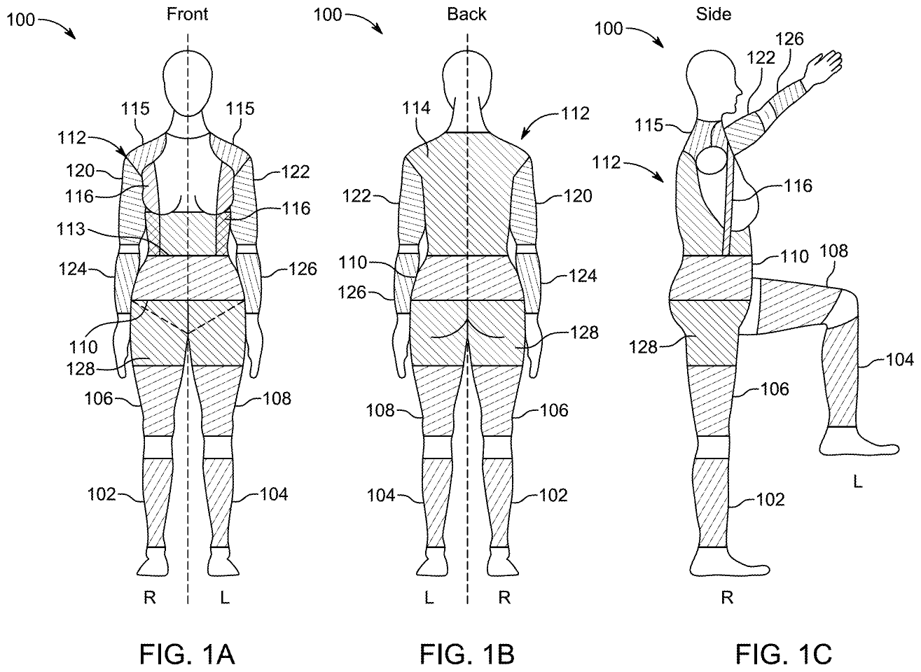

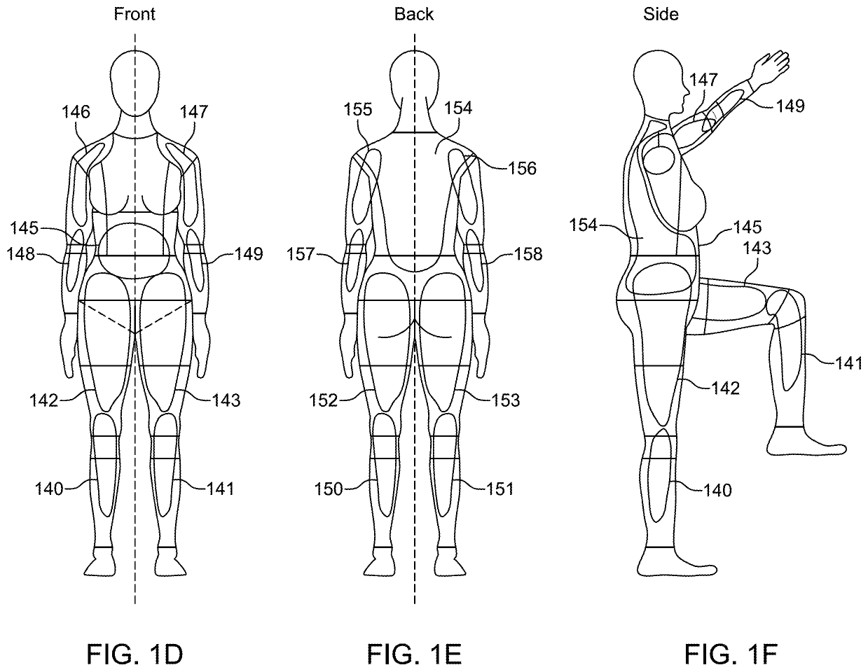

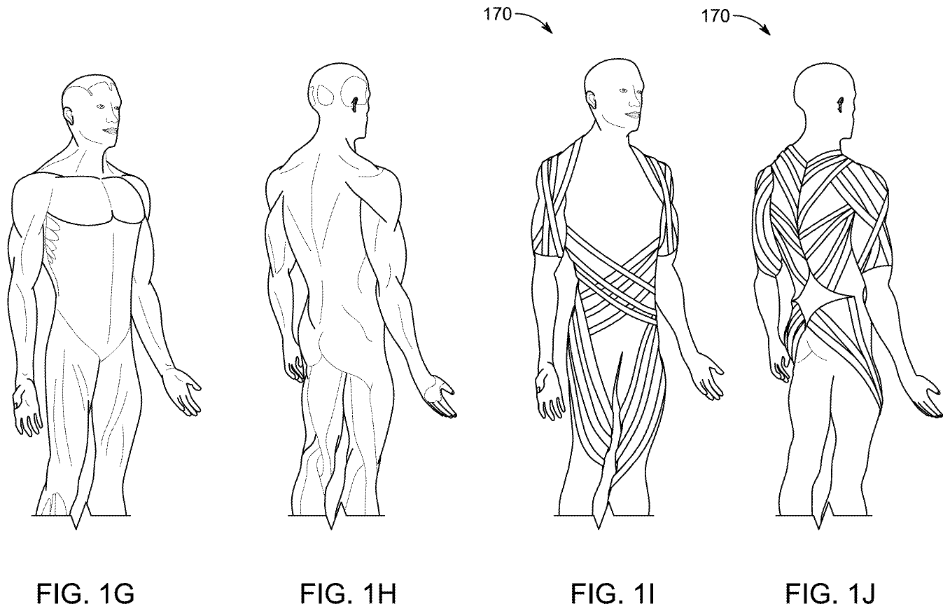

FIGS. 1A-1C show front, back, and side views of a base layer 100 of an exosuit according to an embodiment. Base layer 100 may be worn as a single piece or as multiple pieces. As shown, base layer 100 is shown to represent multiple pieces that can serve as load distribution members (LDMs) for the power layer (shown in FIGS. 1D-1F). Base layer 100 and any LDMs thereof can cover or occupy any part of the human body as desired. The LDMs shown in FIGS. 1A-1C are merely illustrative of a few potential locations and it should be appreciated that additional LDMs may be added or certain LDMs may be omitted.

Base layer 100 can include calf LDMs 102 and 104 that are secured around the calf region or lower leg portion of the human. Calf LDMs 102 and 104 are shown to be positioned between the knees and the ankles, but this is merely illustrative. If desired, calf LDM 102 and 104 can also cover the foot and ankle and/or the knee.

Base layer 100 can include thigh LDMs 106 and 108 that are secured around the thigh region of the human. Thigh LDMs 106 and 108 are shown to be positioned between the knees and an upper region of the thighs. In some embodiments, thigh LDMs 106 and 108 and calf LDMs 102 and 104, respectively, may be merged together to form leg LDMs that cover the entirety of the legs and/or feet.

Base layer 100 can include hip LDM 110 that is secured around a hip region of the human. LDM 110 may be bounded such that it remains positioned above the toileting regions of the human. Such bounding may make toileting relatively easy for the human as he or she would be not be required to remove base layer 100 to use the bathroom. In some embodiments, LDM 110 may be attached to thigh LDMs 106 and 108, but the toileting regions may remain uncovered. In another embodiment, a removable base layer portion may exist between LDM 100 and thigh LDMS 106 and 108.

Base layer 100 can include upper torso LDM 112 that is secured around an upper torso region of the human. Upper torso LDM 112 may include waist LDM 113, back LDM 114, shoulder LDM 115, and shoulder strap LDMs 116. Waist LDM 113, back LDM 114, shoulder LDM 115, and shoulder strap LDMs 116 may be integrally formed to yield upper torso LDM 112. In some embodiments, a chest LDM (not shown) may also be integrated into upper torso LDM 112. Female specific exosuits may have built in bust support for the chest LDM.

Base layer 100 can include upper arm LDMs 120 and 122 and lower arm LDMs 124 and 126. Upper arm LDMs 120 and 122 may be secured around bicep/triceps region of the arm and can occupy space between the shoulder and the elbow. Lower arm LDMs 124 and 126 may be secured around the forearm region of the arm and can occupy the space between the elbow and the wrist. If desired, upper arm LDM 120 and lower arm LDM 124 may be integrated to form an arm LDM, and upper arm LDM 122 and lower arm LDM 126 may be integrated to form another arm LDM. In some embodiments, arm LDMS 120, 122, 124, and 126 may form part of upper torso LDM 112.

Base layer 100 can include gluteal/pelvic LDM 128 that is secured the gluteal and pelvic region of the human. LDM 128 may be positioned between thigh LDMs 106 and 108 and hip LDM 110. LDM 128 may have removable portions such as buttoned or zippered flaps that permit toileting. Although not shown in FIGS. 1A-1C, LDMs may exist for the feet, toes, neck, head, hands, fingers, elbows, or any other suitable body part.

As explained above, the LDMs may serve as attachment points for components of the power layer. In particular, the components that provide muscle assistance movements typically need to be secured in at least two locations on the body. This way, when the flexible linear actuators are engaged, the contraction of the actuator can apply a force between the at least two locations on the body. With LDMs strategically placed around the body, the power layer can also be strategically placed thereon to provide any number of muscle assistance movements. For example, the power layer may be distributed across different LDMs or within different regions of the same LDM to approximate any number of different muscles or muscle groups. The power layer may approximate muscle groups such as the abdominals, adductors, dorsal muscles, shoulders, arm extensors, wrist extensors, gluteals, arm flexors, wrist flexors, scapulae fixers, thigh flexors, lumbar muscles, surae, pectorals, quadriceps, and trapezii.

FIGS. 1D-1F show front, back, and side views, respectively, of a power layer according to an embodiment. The power layer is shown as multiple segments distributed across and within the various LDMs. As shown, the power layer can include power layer segments 140-158. Each of power layer segments can include any number of flexible linear actuators. Some of the power layer segments may exist solely on the anterior side of the body, exist solely on the posterior side, start on the anterior side and wrap around to the posterior side, start on the posterior side and wrap around to the anterior side, or wrap completely around a portion of the body. Power layer segment (PLS) 140 may be secured to LDM 102 and LDM 106, and PLS 141 may be secured to LDM 104 and LDM 108. PLS 142 may be secured to LDM 106 and LDM 110 and/or LDM 114, and PLS 143 may be secured to LDM 108 and LDM 110 and/or LDM 114. PLS 145 may be secured to LDM 110 and LDM 113 and/or to LDM 114 or LDM 128. PLS 146 may be secured to LDM 115 and LDM 120, and PLS 147 may be secured to LDM 115 and LDM 122. PLS 148 may be secured to LDM 120 and LDM 124, and PLS 149 may be secured to LDM 122 and LDM 126.

PLS 150 may be secured to LDM 104 and LDM 108, and PLS 151 may be secured to LDM 102 and LDM 106. PLS 152 may be secured to LDM 106 and LDM 110 and/or to LDM 113, and PLS 153 may be secured to LDM 108 and LDM 110 and/or LDM 113. PLS 154 may be secured to LDM 112 and LDM 110. PLS 155 may be secured to LDM 112 and LDM 120, and PLS 156 may be secured to LDM 112 and LDM 122. PLS 157 may be secured to LDM 120 and LDM 124, and PLS 158 may be secured to LDM 122 and LDM 126.

It should be appreciated that the power layer segments are merely illustrative and that additional power layer segments may be added or that some segments may be omitted. In addition, the attachment points for the power layer segments are merely illustrative and that other attachment points may be used.

The human body has many muscles, including large and small muscles that are arranged in all sorts of different configuration. For example, FIGS. 1G and 1H show respective front and back views of a human male's musculature anatomy, which shows many muscles. In particular, the abdominals, adductors, dorsal muscles, shoulders, arm extensors, wrist extensors, gluteals, arm flexors, wrist flexors, scapulae fixers, thigh flexors, lumbar muscles, pectorals, quadriceps, and trapezii are all shown.

The LDMs may be designed so that they can accommodate different sizes of individuals who don the exosuit. For example, the LDMs may be adjusted to achieve the best fit. In addition the LDMs are designed such that the location of the end points and the lines of action are co-located with the bone structure of the user in such a way that the flexdrive placement on the exosuit system are aligned with the actual muscle structure of the wearer for comfort, and the moment arms and forces generated by the flexdrive/exosuit system feel aligned with the forces generated by the wearer's own muscles.

FIGS. 1I and 1J show front and side views of illustrative exosuit 170 having several power layer segments that approximate many of the muscles shown in FIGS. 1G and 1H. The power layer segments are represented by the individual lines that span different parts of the body. These lines may represent specific flexible linear actuators or groups thereof that work together to form the power layer segments that are secured to the LDMs (not shown). As shown, the FLAs may be arrayed to replicate at least a portion of each of the abdominal muscles, dorsal muscles, shoulder muscles, arm extensor and flexor muscles, gluteal muscles, quadriceps muscles, thigh flexor muscles, and trapezii muscles. Thus, exosuit 170 exemplifies one of many possible different power layer segment arrangements that may be used in exosuits in accordance with embodiments discussed herein. These power layer segments are arranged so that the moment arms and forces generated feel like forces being generated by the user's own muscles, tendons, and skeletal structure. Other possible power layer segment arrangements are illustrated and discussed below.

The power layer segments may be arranged such that they include opposing pairs or groups, similar to the way human muscles are arranged in opposing pairs or groups of muscles. That is, for a particular movement, the opposing pairs or groups can include protagonist and antagonist muscles. While performing the movement, protagonist muscles may perform the work, whereas the antagonist muscles provide stabilization and resistance to the movement. As a specific example, when a user is performing a curl, the biceps muscles may serve as the protagonist muscles and the triceps muscles may serve as the antagonist muscles. In this example, the power layer segments of an exosuit may emulate the biceps and triceps. When the biceps human muscle is pulling to bend the elbow, the exosuit triceps power layer segment can pull on the other side of the joint to resist bending of the elbow by attempting to extend it. The power layer segment can be, for example, either be a FLA operating alone to apply the force and motion, or a FLA in series with an elastic element. In the latter case, the human biceps would be working against the elastic element, with the FLA adjusting the length and thereby the resistive force of the elastic element.

Thus, by arranging the power layer segments in protagonist and antagonist pairs, the power layers segments can mimic or emulate any protagonist and antagonist pairs of the human anatomy musculature system. This can be used to enable exosuits to provide assistive movements, alignment movements, and resistive movements. For example, for any exercise movement requires activation of protagonist muscles, a subset of the power layer segments can emulate activation of antagonist muscles associated with that exercise movement to provide resistance.

The design flexibility of the LDMs and PLSs can enable exosuits to be constructed in accordance with embodiments discussed herein. Using exosuits, the power layer segments can be used to resist motion, assist motion, or align the user's form.

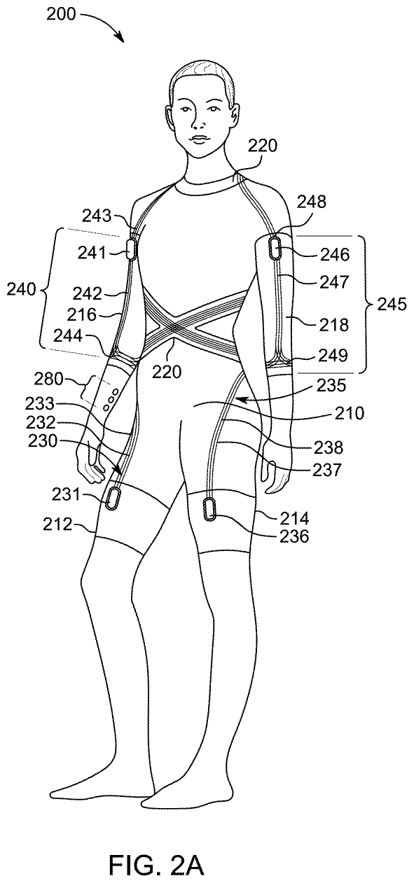

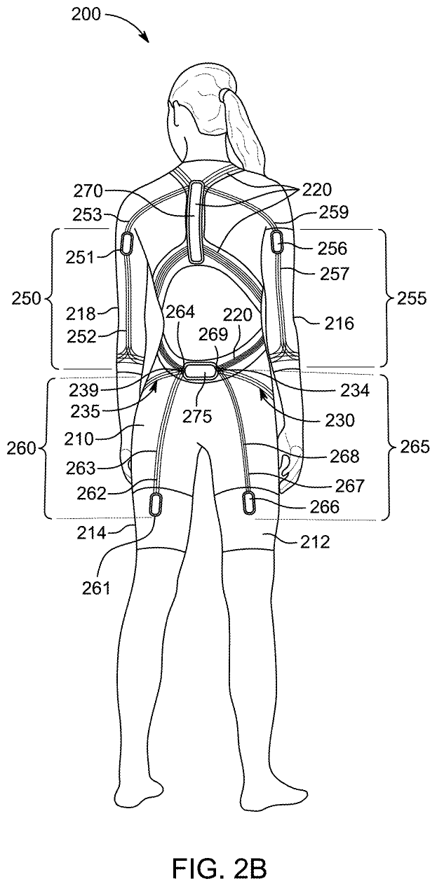

FIGS. 2A and 2B show front and back view of illustrative exosuit 200 according to an embodiment. Exosuit 200 may embody some or all of the base layer, stability layer, power layer, sensor and controls layer, a covering layer, and user interface/user experience (UI/UX) layer, as discussed above. In addition, exosuit 200 may represent one of many different specification implementations of the exosuit shown in FIGS. 1A-F. Exosuit 200 can include base layer 210 with thigh LDMs 212 and 214, arm LDMs 216 and 218, and upper torso LDM 202. Thigh LDMs 212 and 214 may wrap around the thigh region of the human, and arm LDMs 216 and 218 may wrap around arm region (including the elbow) of the human. Upper torso LDM 220 may wrap around the torso and neck of the human as shown. In particular, LDM 220 may cross near the abdomen, abut the sacrum, cover a portion of the back, and extend around the neck.

Exosuit 200 can include extensor PLSs 230 and 235 secured to thigh LDM 212 and 214 and upper torso LDM 220. Extensor PLSs 230 and 235 may provide leg muscle extensor movements. Extensor PLS 230 may include flexdrive subsystem 231, twisted string 232, and power/communication lines 233. Flexdrive subsystem 231 may include a motor, sensors, a battery, communications circuitry, and/or control circuitry. Twisted string 232 may be attached to flexdrive subsystem 231 and an attachment point 234 on LDM 220. Power/communications lines 233 may convey control signals and/or power to flexdrive subsystem 231. Extensor PLS 235 may include flexdrive subsystem 236, twisted string 237, and power/communication lines 238. Twisted string 237 may be attached to flexdrive subsystem 236 and attachment point 239.

Exosuit 200 can include flexor PLSs 240 and 245 and extensor PLSs 250 and 255 that are secured to LDMs 216, 218, and 220 (as shown). Flexor PLSs 240 and 245 may provide arm muscle flexor movements, and extensor PLSs 250 and 255 may provide arm muscle extensor movements. Flexor PLS 240 may include flexdrive subsystem 241, twisted string 242, and power/communication lines 243. Twisted string 242 may be attached to flexdrive subsystem 241 and attachment point 244. Power/communication lines 243 may be coupled to power and communications module 270. Flexor PLS 245 may include flexdrive subsystem 246, twisted string 247, and power/communication lines 248. Twisted string 247 may be attached to flexdrive subsystem 246 and attachment point 249. Power/communication lines 248 may be coupled to power and communications module 270. Extensor PLS 250 may include flexdrive subsystem 251, twisted string 252, and power/communication lines 253. Twisted string 252 may be attached to flexdrive subsystem 251 and attachment point 254. Power/communication lines 253 may be coupled to power and communications module 270. Extensor PLS 250 may include flexdrive subsystem 256, twisted string 257, and power/communication lines 258. Twisted string 256 may be attached to flexdrive subsystem 256 and attachment point 259. Power/communication lines 258 may be coupled to power and communications module 270.

Exosuit 200 can include flexor PLS 260 and 265 that are secured to thigh LDMs 212 and 214 and LDM 220. Flexor PLSs 260 and 265 may provide leg muscle flexor ARA movements. Flexor PLS 260 may include flexdrive subsystem 261, twisted string 262, and power/communication lines 263. Twisted string 262 may be attached to flexdrive subsystem 261 and attachment point 264. Power/communication lines 263 may be coupled to power and communications module 275. Flexor PLS 266 may include flexdrive subsystem 266, twisted string 267, and power/communication lines 268. Twisted string 267 may be attached to flexdrive subsystem 266 and attachment point 269. Power/communication lines 263 may be coupled to power and communications module 275

Exosuit 200 is designed to assist, resist, and align movements being performed by the user of the suit. Exosuit 200 may include many sensors in various locations to provide data required by control circuitry to provide such movements. These sensors may be located anywhere on base layer 210 and be electrically coupled to power and communications lines (e.g., 233, 237, 243, 247, 253, 257, 263, 267, or other lines). The sensors may provide absolute position data, relative position data, accelerometer data, gyroscopic data, inertial moment data, strain gauge data, resistance data, or any other suitable data.

Exosuit 200 may include user interface 280 that enables the user to control the exosuit. For example, user interface 280 can include several buttons or a touch screen interface. User interface 280 may also include a microphone to receive user spoken commands. User interface 280 may also include a speaker that can be used to playback voice recordings. Other user interface element such as buzzers (e.g., vibrating elements) may be strategically positioned around exosuit 200.

Exosuit 200 can include communications circuitry such as that contained in power and communications module 270 or 275 to communicate directly with a user device (e.g., a smartphone) or with the user device via a central sever. The user may use the user device to select one or more movements he or she would like to perform, and upon selection of the one or more movements, exosuit 200 can the assist, resist, or align movement. The user device or exosuit 200 may provide real-time alignment guidance as to the user's performance of the movement, and exosuit 200 may provide resistance, alignment, or assistance to the movement.

An exosuit can be operated by electronic controllers disposed on or within the exosuit or in wireless or wired communication with the exosuit. The electronic controllers can be configured in a variety of ways to operate the exosuit and to enable functions of the exosuit. The electronic controllers can access and execute computer-readable programs that are stored in elements of the exosuit or in other systems that are in direct or indirect communications with the exosuit. The computer-readable programs can describe methods for operating the exosuit or can describe other operations relating to a exosuit or to a wearer of a exosuit.

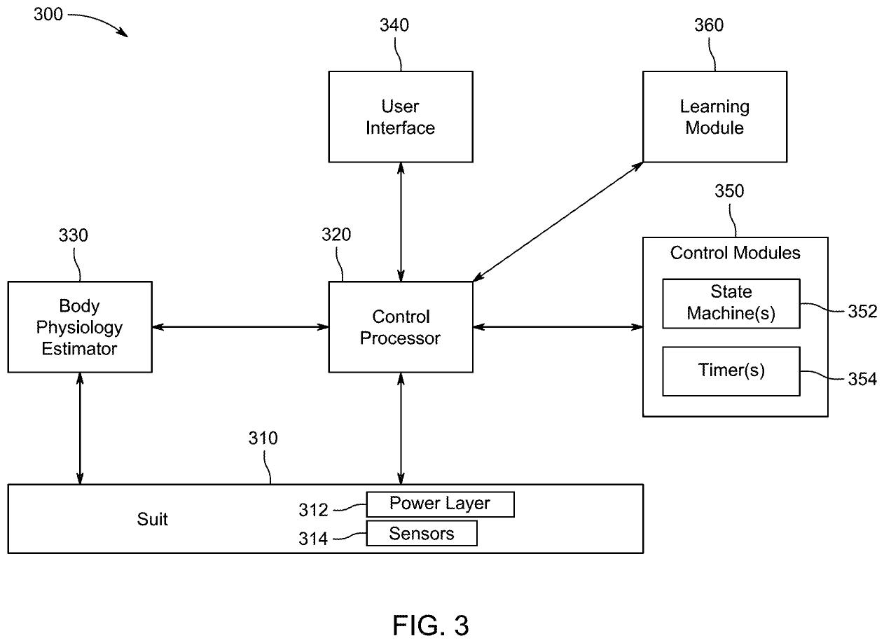

FIG. 3 shows an illustrative symbiosis exosuit system 300 according to an embodiment. Symbiosis is generally a feature where the exosuit is measuring the posture of the wearer and automatically moving to support the wearer based solely on triggers, that may be body motions, timers, or even external measures. Symbiosis enables the exosuit to automatically do what the wearer expects, sort of like an artificial autonomic motor nervous system. The symbiosis enables the exosuit to serve as an autonomous exosuit nervous system that mimics or emulates the nervous system of a lifeform such as a human being. That is, a nervous system is responsible for basic life functions (e.g., breathing, converting food into energy, and maintaining muscle balance) that are performed automatically without requiring conscious thought or input. The autonomous exosuit nervous system enables the exosuit to automatically provide assistance to the user when and where the user needs it without requiring intervention by the user. Exosuit system 300 can do this by tracking the user's body physiology and automatically controlling the suit to provide the anticipated or required support and/or assistance. For example, if a user has been standing for a prolonged period of time, one or more of the muscles being used to help the user stand may begin to tire, and a result, the user's body may exhibit signs of fatigue. Exosuit 300 can observe this muscle fatigue (e.g., due to observed physiological signs) and can automatically cause exosuit 300 to engage the appropriate power layers to compensate for the muscle fatigue.

Symbiosis of exosuit 300 may be expressed in different autonomy levels, where each autonomy level represents a degree to which physiological factors are observed and a degree to which suit assistance or movement actions are performed based on the observed physiological factors. For example, the symbiosis levels can range from a zero level of autonomy to absolute full level of autonomy, with one or more intermediate levels of autonomy. As metaphorical example, autonomous cars operate according to different levels, where each level represents a different ability for the car to self-drive. The symbiosis levels of exosuit operation can be stratified in a similar manner. In a zero level of autonomy, exosuit 300 may not monitor for any physiological cues, nor automatically engage any suit assistance or movement actions. Thus, in a zero level, the user may be required to provide user input to instruct the suit to perform a desired movement or assistance. In an absolute full level of autonomy, exosuit 300 may be able to observe and accurately analyze the observed physiological data (e.g., with 99 percent accuracy or more) and automatically execute the suit assistance or movement actions in a way expressly desired by the user. Thus, in the absolute full level, the exosuit seamlessly serves as an extension of the user's nervous system by automatically determining what the user needs and providing it.

The one or more intermediate levels of autonomy provide different observable physiological results that are accurate but do not represent the absolute nature of the absolute full level of autonomy. For example, the intermediate levels may represent that the exosuit is fully capable of autonomously performing certain actions (e.g., sit to stand) but not others. A corollary to this is ABS braking; the ABS braking system automatically figures out how best to stop the vehicle without requiring the user to pump the brakes or engage in any other activity other than stepping on the brake pedal. In the exosuit context, the exosuit knows when the user wishes to stand from a sitting position, the exosuit knows when the user wishes to perform the movement and engages the appropriate power layer segments to assist in the movement. The intermediate levels may also exist while the exosuit is learning about its user. Each user is different, and the physiological responses are therefore different and particular to each user. Therefore, the ability to discern the physiological cues and the assistance and movements made in response thereto may endure a learning curve before the suit is able to operate at the absolute full level.

FIG. 3 shows that exosuit system 300 can include suit 310, control processor 320, body physiology estimator 330, user interface 340, control modules 350, and learning module 360. Suit 310 can be any suitable exosuit (e.g., exosuit 200) and can include, among other things, power layer 312 and sensors 314. Control processor 320 may process instructions, pass data, and control the suit. Control processor 320 may be connected to suit 310, body physiology estimator 330, user interface 340, control modules 350, and learning module 360. Control processor 320 may provide signals to suit 310 to control, for example, operation of power layer 312.

Body physiology estimator 330 may receive data inputs from sensor 314, control processor 320, and other components if desired. Estimator 330 can sometimes be referred to as an activity classifier. Estimator 330 is operative to analyze the data to ascertain the physiology (e.g., body tone or body posture) of the user. Estimator 330 may apply data analytics and statistics to the data to resolve physiological conditions of the user's body. For example, estimator 330 can determine whether the user is sitting, standing, leaning, laying down, laying down on a side, walking, running, jumping, performing exercise movements, playing sports, reaching, holding an object or objects, or performing any other static or active physiological event. The results may be provided to control modules 350, for example, via control processor 320. In the context of symbiosis, estimator 330 automatically tracks the posture of the wearer and evaluates when a posture is identified that is associated with an assistance motion. The assistance motions are activated with a base tone to a ready-to-assist position, and then full assistance when the suit assist trigger is identified for that posture. The trigger is a movement or other indicator (i.e., a timer) that is related to the posture and the action it initiates.

Control modules 350 can include various state machines 352 and timers 354 operative to control operation of suit 310 based on outputs supplied by estimator 330, inputs received via user interface 340, and signals provided by control processor 320. Multiple state machines 352 may control operation of the suit. For example, a master state machine may be supported by multiple slave state machines. The slave state machines may be executed in response to a call from the master state machine. In addition, the slave state machines may execute specific assistance functions or movements. For example, each of a sit-to-stand assistance movement, stand-to sit movement, stretch movement, standing movement, walking movement, running movement, jumping movement, crouch movement, specific exercise movement, or any other movement may have its own slave state machine to control suit operation.

Learning module 360 may be operative to learn preferences, peculiarities, or other unique features of a particular user and feedback the learnings to body physiology estimator 330 and control module 350. In some embodiments, learning module 360 may use data analytics to learn about the user. For example, learning module 360 may learn that a particular user walks with a particular gait and cadence. The gait and cadence learnings can be used to modify state machines 352 that control walking for that user. In another embodiment, learning module 360 may incorporate user feedback received via user interface 340. For example, a user may go through an initial setup process whereby the user is instructed to perform a battery of movements and provide responses thereto so that state machines 352 and timers 354 are set to operate in accordance with the preferences of the user.

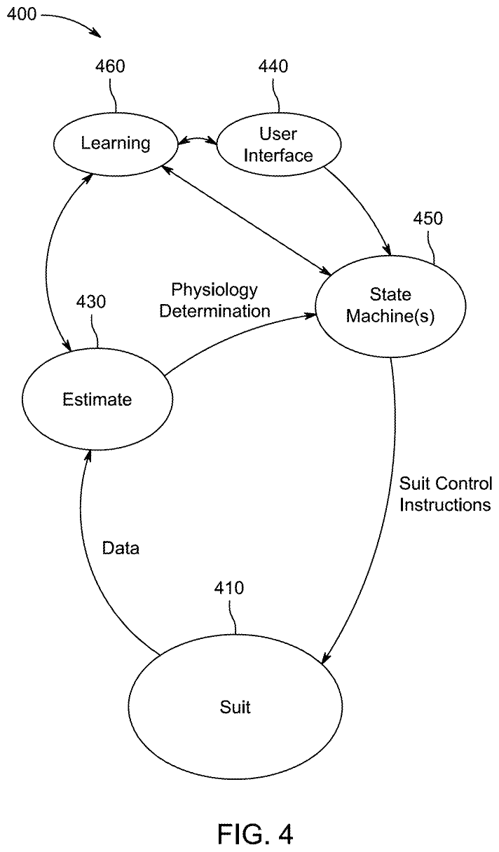

FIG. 4 shows illustrative process 400 for implementing symbiosis exosuit system 300 according to an embodiment. Process 400 includes suit 410, estimator 430, user interface 440, and state machines 450. Process 400 can be represented by a continuous feedback loop in which data is supplied from suit 410 to estimator 430, which provides a physiology determination to state machines 450, which uses the determination to generate suit control instructions that are provided to suit 410. User inputs received via user interface 440 may provide user specified controls that can instruct state machines 450 to execute a particular movement. The autonomous exosuit nervous system is implemented through the continuous feedback loop. The continuous feedback loop enables the autonomous exosuit nervous system to provide rapid response and control of suit 410. For example, if the user is sitting down, the estimator 430 can determine that the sitting position is the current physiological determination. Assume that the user reaches for something on a table. Such a movement may result in a movement that appears to be a sit-to-stand. In response to this movement, estimator 430 may register it as the start of a sit-to-stand physiological determination and instruct state machines 450 to initiate a sit-to-stand movement. This way, regardless of whether the user actually stands or sits back down, suit 410 is primed and ready to immediately perform the assistance movement. Further assume that the user sits back down (after having grabbed the item on the table). In response to initiation of the sit down movement, estimator 430 can make this determination as it is happening and instruct state machines 450 to cease the sit-to-stand operation. Thus, the continuous feedback loop provides real-time assessment and instantaneous suit controls in response to the user's immediate physiological needs, and not after.

In some embodiments, estimator 430 may be able to determine that the user is was attempting to reach something on the table while also performing the motion that includes at least the start of a sit to stand movement. Estimator 430 may be able to correlate the reaching motion with the sit-to-stand motion and decide that the user does not actually need to stand, but may require an appropriate amount of assist to reach the item. In this particular situation, state machine 450 may activate a power layer segment (e.g., a particular one of the hip extensors) to provide the user with the reach assistance.

Learning 460 can receive and provide data to estimator 430, user interface 440, and state machines 450. Learning 460 may be leveraged to update state machines 450 and/or estimator 430.

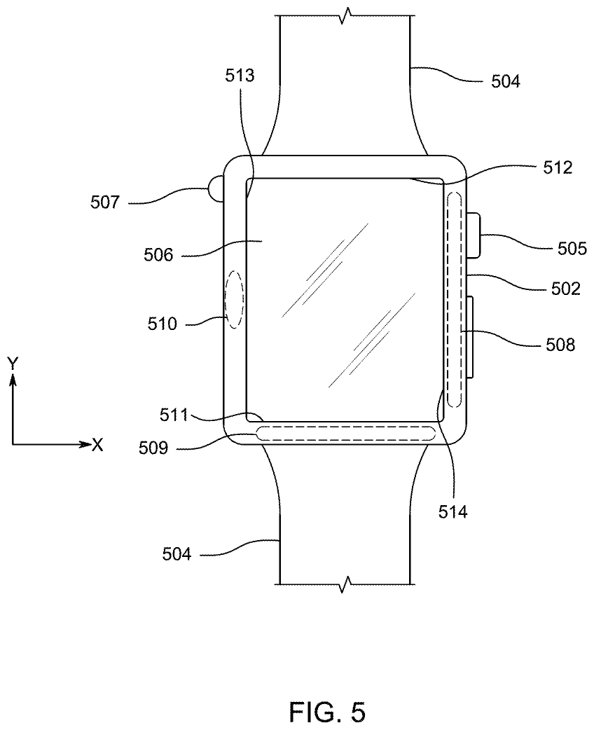

FIG. 5 illustrates exemplary personal electronic device 500 that may be used in connection with an exosuit. In the illustrated example, device 500 is a watch that generally includes housing 502 and band assembly or strap 504 for affixing device 500 to the body of a user. That is, device 500 is wearable. Housing 502 can be designed to couple with straps 504. Device 500 can have dial 505, touch-sensitive display screen (hereafter touchscreen) 506, buttons 507, capacitive interface regions 508-510 provided by regions of housing 502. Housing 502 may take any suitable shape, including, for example, a rectangular cuboid shape or a cylindrical shape.

Capacitive interface regions 508-510 may be regions of housing 502 in which a user can perform touch gestures on housing 502 to interact with content displayed on touchscreen 506 without having to touch touchscreen 506. Capacitive interface regions 508-510 can occupy different sized areas on housing 502. In some embodiments, capacitive interface regions 508-510 can mimic a contour of housing 502 and/or a shape of at least a portion of a border or edge or side or periphery of touchscreen 506. For example, capacitive interface region 508, which may be provided by a portion of a front face or surface of housing 502 positioned to the right of touchscreen 506, may span adjacent to the length of the right touchscreen border or edge or side or periphery 514 of touchscreen 506 along the y-axis from bottom touchscreen border or edge or side or periphery 511 to top touchscreen border or edge or side or periphery 512.

Dial 505 may be rotated, depressed, or pulled out to perform various different inputs. Buttons 507 (only one is shown) may be pressed by the user to provide inputs to control the user interface.

Display 506 can include any suitable display device, such as a liquid crystal display (LCD), light-emitting diode (LED) display, organic light-emitting diode (OLED) display, or the like, positioned partially or fully behind or in front of a touch sensor panel implemented using any desired touch sensing technology, such as mutual-capacitance touch sensing, self-capacitance touch sensing, resistive touch sensing, projection scan touch sensing, or the like. Display 506 can allow a user to perform various functions by touching over hovering near the touch sensor panel using one or more fingers or other objects.

In some examples, device 500 can further include one or more pressure sensors (not shown) for detecting an amount of force or pressure applied to the display. The amount of force or pressure applied to display 506 can be used as an input to device 500 to perform any desired operation, such as making a selection, entering or exiting a menu, causing the display of additional options/actions, or the like. In some examples, different operations can be performed based on the amount of force or pressure being applied to display 506. The one or more pressure sensors can further be used to determine a position on which a force is being applied to display 506.

It should be understood that device 500 is one example of an accessory device that may be used in connection with one or more exosuits according to various embodiments discussed herein. For example, the accessory device can be embodied in other devices such as tablets, smartphones, laptops, desktops, etc. Furthermore, the embodiments discussed herein do not necessarily have to be implemented on a device such as device 500, but can be implemented in a computer application or web application running on any suitable device. In some embodiments, a single accessory device may be used to communicate with multiple exosuit systems.

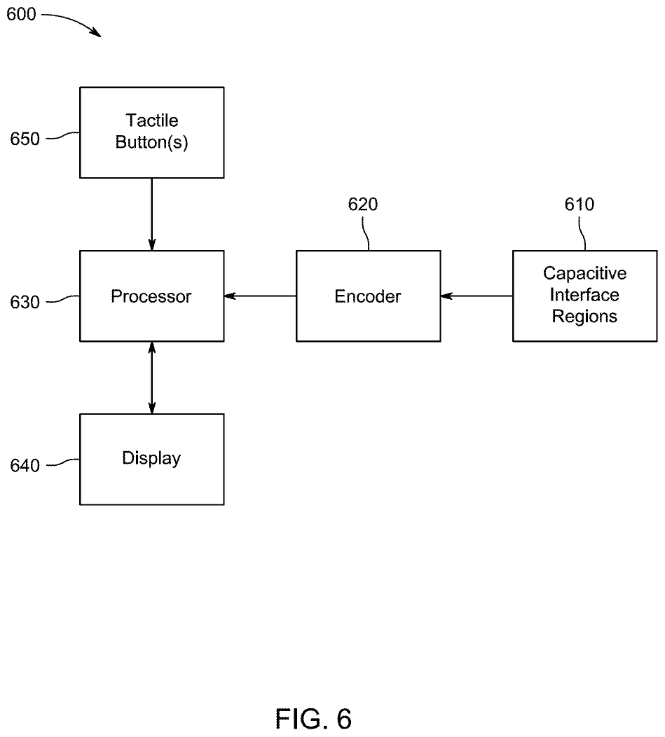

FIG. 6 illustrates a block diagram of some of the components of a device 600, which may be similar to device 500 according to some embodiments. As shown, one or more capacitive interface regions 610 can be coupled to encoder 620, which can be configured to process touch events received on each interface region 610, and to provide electrical signals representative of the touch events to processor 630. Encoder 620 can be configured to process a variety of touch events on capacitive interface regions 610. Encoder 620 can detect single touch events, double touch events, extended touch events, scroll direction events and associated speed of the scroll (along one or more axes), multi-touch events (e.g., zoom and coordinate-based selections), and any other suitable touch events. Encoder 620 can sense the absolute touch position anywhere within an interface region. Encoder 620 can be configured to sense a direction of a touch event. Encoder 620 can be configured to detect a speed of touch events on regions 610 in any desired manner (e.g., velocity, acceleration, or the like) and can provide the speed information to processor 630. The speed can be expressed in numerous ways. For example, the speed can be expressed in a direction and a speed, such as hertz, as distance versus time, as a change in angle per unit of time, and the like. In alternative examples, instead of providing information to processor 630, this information can be provided to other components of device 600. While the examples described herein refer to the use of touch events on interface regions 610 to control user interaction with content on a screen, it should be appreciated that any other inputs derived from interface regions 610 can be used.

In some examples, the touch inputs received via interface regions 610 can control physical attributes of content displayed on display 640 of device 600. For example, if a user scrolls his finger in a y-axis direction along interface region 610, display 640 may show content being scrolled in the same y-axis direction of the user. In other words, the physical touch inputs received by interface regions 610 can represent physical modal functionality of display 640. In some examples, a temporal attribute of a user touch input on interface region 610 can be used as an input to device 600. For example, a fast change in touch inputs can be interpreted differently than a slow change in touch inputs.

Processor 630 can be further coupled to receive input signals from tactile or mechanical buttons 650, along with touch signals from touch-sensitive display 630, and/or signals received from a remote device such as a user's phone or laptop. Processor 620 can be configured to interpret these input signals and output appropriate display signals to cause an image to be produced by touch-sensitive display 630. While a single processor 630 is shown, it should be appreciated that any number of processors or other computational devices can be used to perform the general functions discussed above.

Embodiments discussed herein refer to systems and methods for controlling and interacting with an exosuit using a personal device such as device 500. The personal device can be any suitable device that communicates with the exosuit via a wireless or wired connection. A user can interact with the personal device to control the exosuit. For example, the user can initiate various exosuit functions such as standing support, lumbar support, a sit-to-stand operation, walking, etc. The user can cancel any action using the personal device. The user can adjust various exosuit settings using the personal device. For example, the user can adjust the tension of each FLA. In the following discussion, several embodiments for exosuit control via a personal device are discussed.

As defined herein, basetone may refer to a “ready” state in which the exosuit's power layers, including one or more FLAs, have engaged to apply a predefined tension force that is aligned with a possible exosuit assistance action that will support any possible movements that the user of the exosuit could make. The basetone can alert the user of the exosuit that the suit is ready to make an assistance movement and also primes the power layers to a state to quickly apply force so that the user does not have to wait for the power layers to get up to speed (e.g., take up the slack in the FLAs) to execute the assistance movement. That is, the user can feel the tension/contraction of the power layer(s) primed for execution of an assistance feature. In practice, the basetone can increase perceived responsiveness of the exosuit as it initiates an action. Basetone may be performed as a background operation that does not require active intervention by a central command processor, a personal device, or the user. In other words, the user may not necessarily be aware of the self-adjusting being performed by the exosuit. In other words, the exosuit can implement the self-adjusting functions without unduly restricting movement of the user or producing too much noise. In some embodiments, the exosuit's basetone may also provide muscle assistance based on a determination that the user requires such assistance. During an initial setup of the exosuit, the user may choose an initial basetone setting that may serve as the baseline for the basetone. Thereafter, the user may adjust the basetone to have to a different baseline as desired.

As defined herein, body language refers to ways in which body motion is communicated to the exosuit. In other words, the sensors located on the exosuit may detect the position and movement of the person wearing the suit.

As defined herein, lumbar support refers to contraction of one or more power layers to provide support for the lower back. The lumbar support may be provided independent of any other support features.

As defined herein, micro-adjust refers to the ability to increase or decrease tension or force in controlled increments.

As defined herein, sit to stand support refers to suit assist for sit to stand assistance feature.

As defined herein, standing support refers to contraction of hip flexor and extensor power layers to help pull the user into proper posture. Standing support can also add assistance strength and/or stability in standing.

As defined herein, body tone refers to the physiology of the user of the exosuit. This can include a force state of a particular muscle. For example, body tone can refer to the muscle stiffness or tightness in a fixed position. The human body has a certain body tone in a certain posture.

As defined herein, body posture can refer to the posture of the wearer of the exosuit. The body posture can be defined as the body orientation that is related to a functional activity (e.g., sitting, standing, kneeling, etc.). In some embodiments discussed herein, body tone may be replaced with body posture, and vice versa.

As defined herein, trigger refers to any event that sends a command to the exosuit. For example, a trigger can be implemented as body language, a wireless command received from a personal device (e.g., device 500), or a timer based command received from a personal device.

As defined herein, undo or cancel can refer to a command to cancel or stop any assistance action, support action, or micro-adjustment.

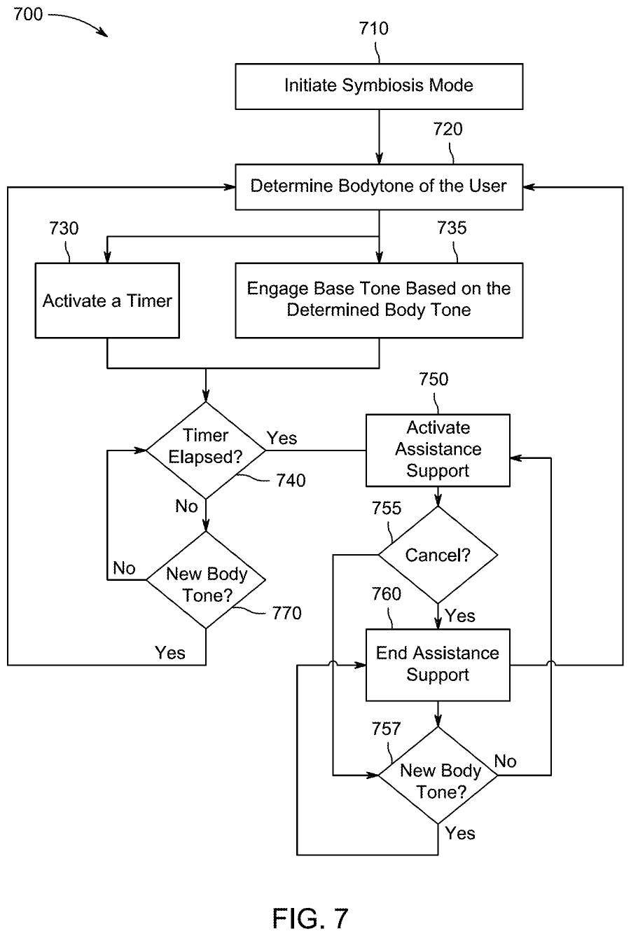

FIG. 7 shows illustrative process 700 for implementing timer based symbiosis of an exosuit according to an embodiment. Starting at step 710, a symbiosis mode may be initiated. The symbiosis mode can invoke process 400 of FIG. 4 and enable the exosuit to automatically detect the physiology of the user and take actions in accordance with one or more state machines. In the context of process 700, the symbiosis mode is being used in combination with a timer to control whether an assistance action should be activated. At step 720, a body tone or body posture of the user can be determined. The body tone or body posture can be a physiological determination of the user's positional state and/or predicted motion state. For example, the body tone may indicate that the user is standing. Subsequent to step 720, a timer may be activated at step 730 and a base tone may be engaged based on the determined body tone, at step 735. The timer may be countdown timer. The duration of the countdown timer may be set based on the determined body tone or a user preference setting. Engagement of the base tone may result in pre-emptive activation of one or more power layers that will be used to activate assistance support (e.g., lumbar support). At step 740, a determination is made if the timer has elapsed. If YES, process 700 can proceed to step 750, which may activate the assistance support. At step 755, a determination is made as to whether a cancel action has been received. A cancel action may be initiated by a user via his or her personal device. If the determination at step 755 is YES, the assistance support is ended at step 760. If the determination at step 755 is NO, process 700 can proceed to step 757. At step 757, a determination is made as to whether the user exhibits a new body tone. For example, if the user has been standing, and then decides to sit down, the body tone is no longer in the standing position. If at step 757, there is NO new body tone, process 700 loops back to step 750. If at step 757, there is a new body tone, process 700 proceeds to step 760. After step 760, process 700 loops back to step 720.

If the timer has not yet elapsed at step 740, process 700 may determine whether a new body tone exist at step 770. If the determination at step 770 is NO, process 700 may loop back to step 740. If the determination at step 770 is YES, process 700 may loop back to step 720.

It should be appreciated that the steps shown in FIG. 7 are merely illustrative and that additional steps may be added and the order in which steps are executed can be changed. For example, the user cancels symbiosis mode at any time at which point, all activity, including base tone operation ceases. As another example, more than one assistance support can be active at the same time. A specific example is that a combination of standing and lumbar support can be provided. Thus, if a user determined to have a standing body tone, the base tones for lumbar and standing support can be activated, and after the timer elapses (and the user is still in the standing body tone), the both lumbar and standing support modes may be activated. If the user decides to start walking, standing support may be disengaged, but lumbar support may remain engaged.

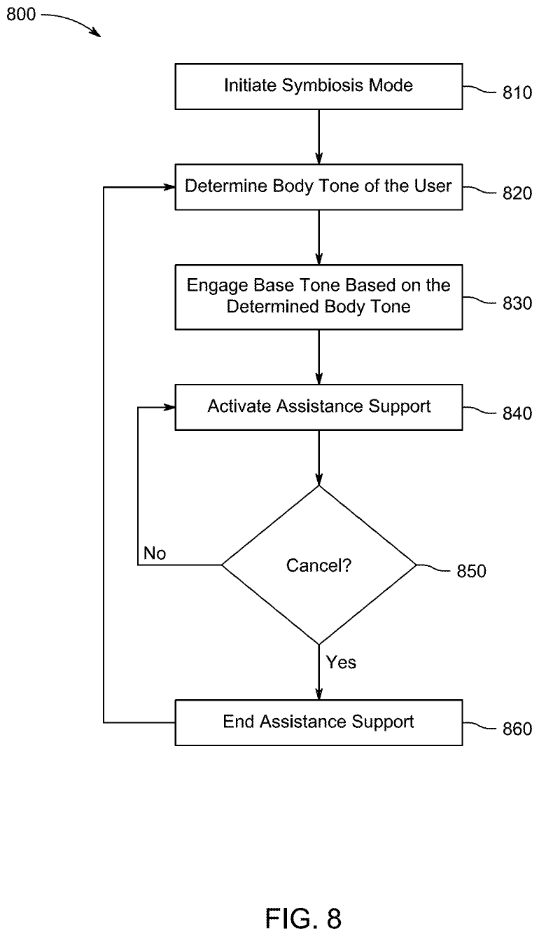

FIG. 8 shows illustrative process 800 for implemented timer free symbiosis modes according to an embodiment. Starting at step 810, a symbiosis mode may be initiated. At step 820, a body tone of the user can be determined. Engagement of the base tone based on the determined body tone may occur at step 830. At step 840, assistance support can be activated. At step 850, a determination is made as to whether a cancel action has been received. If the determination at step 840 is NO, process 800 can loop back to step 840. If the determination at step 840 is YES, the assistance support may end at step 860 and process 800 may loop back to step 820.

One example implementation of process 800 may involve a sit-to-stand operation. At step 820, the body tone may indicate that the user is getting ready to stand. In response to this indication, the exosuit may engage the sit-to-stand base tone in preparation for the sit-to-stand assistance support. When the user actually commences with standing, the exosuit activates the appropriate power layers to perform the sit-to-stand operation.

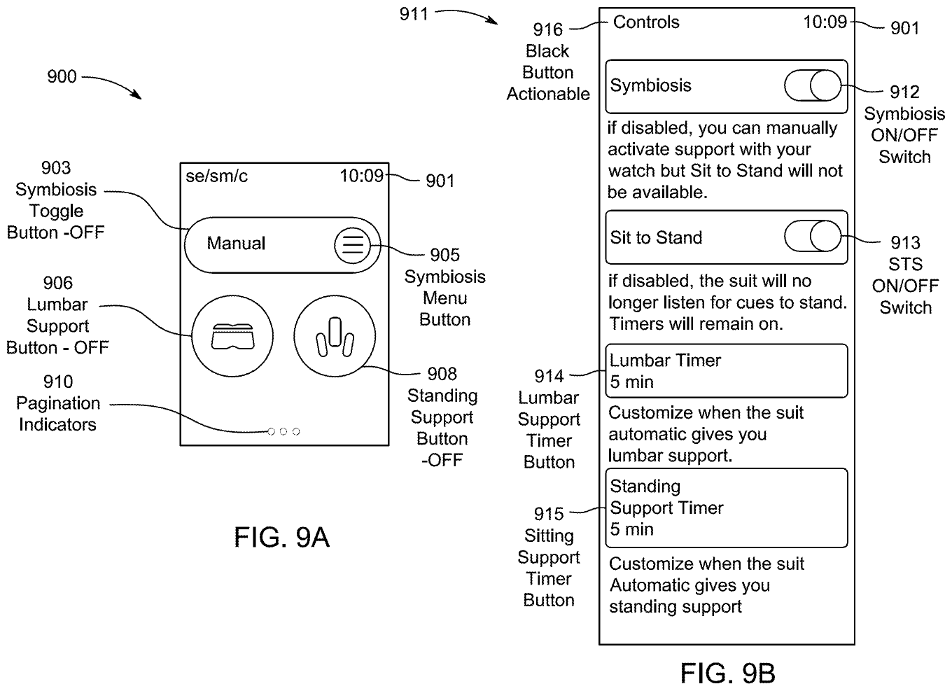

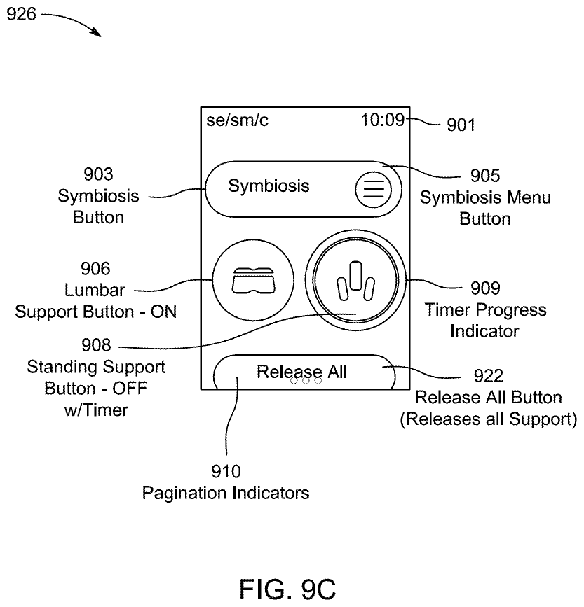

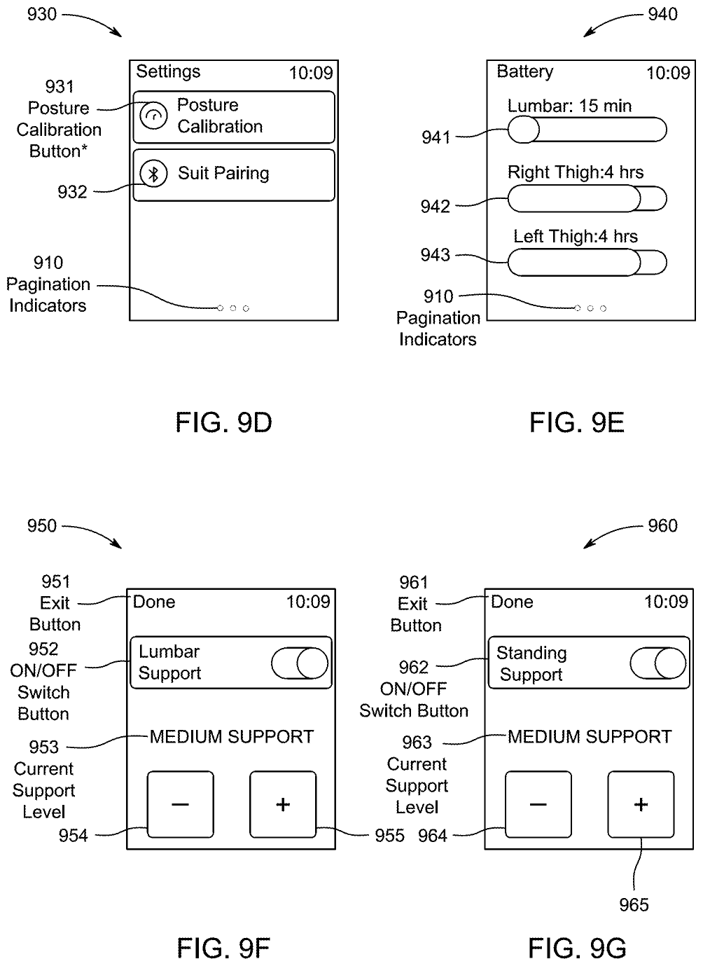

FIGS. 9A-9G shows several different personal device screens according to various embodiments. The screens show different user selectable icons and submenus that may be manipulated to change different features of an exosuit. Starting with FIG. 9A, control screen 900 can include time 901, symbiosis toggle button 903, symbiosis menu button 905, lumbar support button 906 (shown as OFF), standing support button 908 (shown as OFF), and pagination indicator 910. A user may select any of buttons 903, 906, and 908 to toggle that feature ON and OFF. A user may access other pages by swiping left and/or right on screen 900 (and the appropriate pagination indicator 910 would be illuminated). Symbiosis toggle button 903 may enable to operate the exosuit in a manual mode or a symbiosis mode. In the manual mode, the user may be required to manually activate support assistance by selecting the appropriate button (e.g., button 906 or button 908).

When the user selects button 905, symbiosis screen 911 of FIG. 9B may be presented. Screen 911 shows time 901, symbiosis on/off switch 912, sit-to-stand on/off switch 913, lumbar timer 914, standing support timer 915, and back button 916. ON/OFF switches 912 and 913 are self-explanatory. If user selects timers 914 or 915, he can set the timer to any desired value or to a value within a fixed range of values. The timer can indicate how much time must elapse before the support assistance is provided. Alternatively, the timer can indicate how long the support is provided before it is turned off.

FIG. 9C shows control screen 920 with symbiosis toggle button 902 in the ON position, lumbar support button 906 (shown as ON), standing support button 908 (shown as OFF) but with count down timer 909, pagination indicator 910, and release button 922. Count down timer 909 may be a visual indicator showing how much time is left before the standing support assistance is activated. Release button 922 may cause all assistance functions to cease operating when pressed.

FIG. 9D shows settings screen 930 with posture calibration button 931 and suit pairing button 932. A user may press button 931 to calibrate the suit (as discussed in further detail below). A user may press button 932 to pair an exosuit to the personal device.

FIG. 9E shows battery status screen 940 showing remaining power for various power layers. Screen 940 can include lumbar status 941, right thigh status 942, and left thigh status 943. Other status regions can be shown as desired.

FIG. 9F shows lumbar support screen 950 showing exit button 951, ON/OFF switch 952, support level status 953, decrease support button 954, and increase support button 954. The user can turn lumbar support ON or OFF via switch 952. Status 953 can indicate the current level of support being provided by the lumbar power layer. The user can decrease or increase the level of support provided by pressing buttons 954 or 955. Status 953 can change in response to presses of buttons 954 and 955.

FIG. 9G shows standing support screen 960 showing exit button 961, ON/OFF switch 962, support level status 963, decrease support button 964, and increase support button 964. The user can turn standing support ON or OFF via switch 962. Status 963 can indicate the current level of support being provided by the lumbar power layer. The user can decrease or increase the level of support provided by pressing buttons 964 or 965. Status 963 can change in response to presses of buttons 964 and 965.

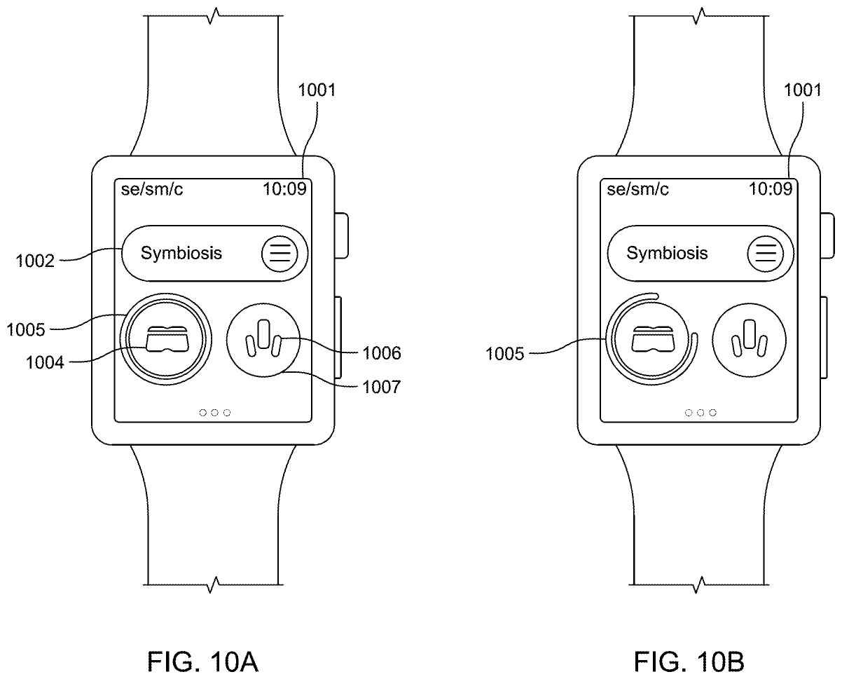

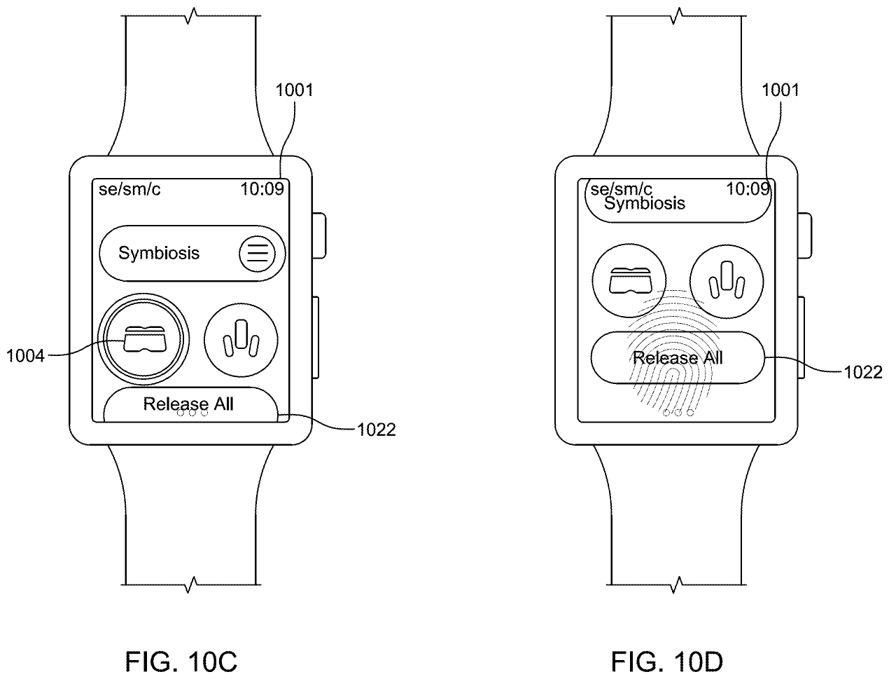

FIGS. 10A-10D show different screens that may be shown as part of lumbar support via timer according to an embodiment. FIG. 10A shows screen 1001 that include symbiosis ON/OFF button 1002, lumbar support button 1004 with timer 1005, and standing support button 1006 with timer 1007. Screen 1001 indicates symbiosis mode is ON. In FIG. 10B, the exosuit may recognize that the user is in the standing position and can initiate countdown timer 1005, as shown by the circular progress bar. If the user remains in the standing position and countdown timer 1005 lapses, lumbar support assistance may be initiated. FIG. 10C shows that lumbar support is initiated by changing the color or background of lumbar support button 1004. Screen 1001 may now present release button 1022. In FIG. 10D, the user is shown pressing button 1022 to cease lumbar support assistance.

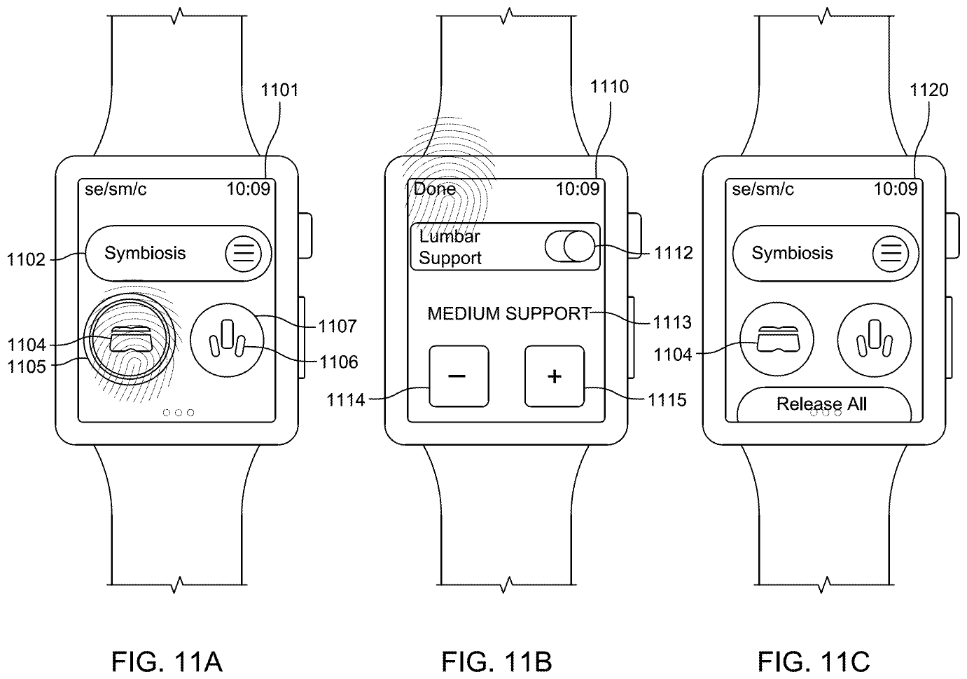

FIGS. 11A-11C show different screens that may be shown as part of manually activated lumbar support according to an embodiment. FIG. 11A shows screen 1101 having symbiosis ON/OFF button 1102, lumbar support button 1104 with timer 1105, and standing support button 1106 with timer 1107 (not shown). Screen 1101 shows timer 1105 is present, but has not elapsed. Screen 1101 also shows symbiosis button 1101 is ON. The user may select lumbar support button 1104 to turn ON lumber support assistance prior to lapse of the timer. If the user presses and hold on to button 1104, screen 1110 of FIG. 1B may be shown. Screen 1110 is a settings screen for lumbar support and includes lumbar support ON/OFF button 1112, support level status 1113, decrease button 1114 and increase button 1115. If the user presses done button 1117, screen 1120 of FIG. 11C may be presented. Screen 1120 shows that lumbar support button 1104 is in the ON position and release button 1122 is also provided.

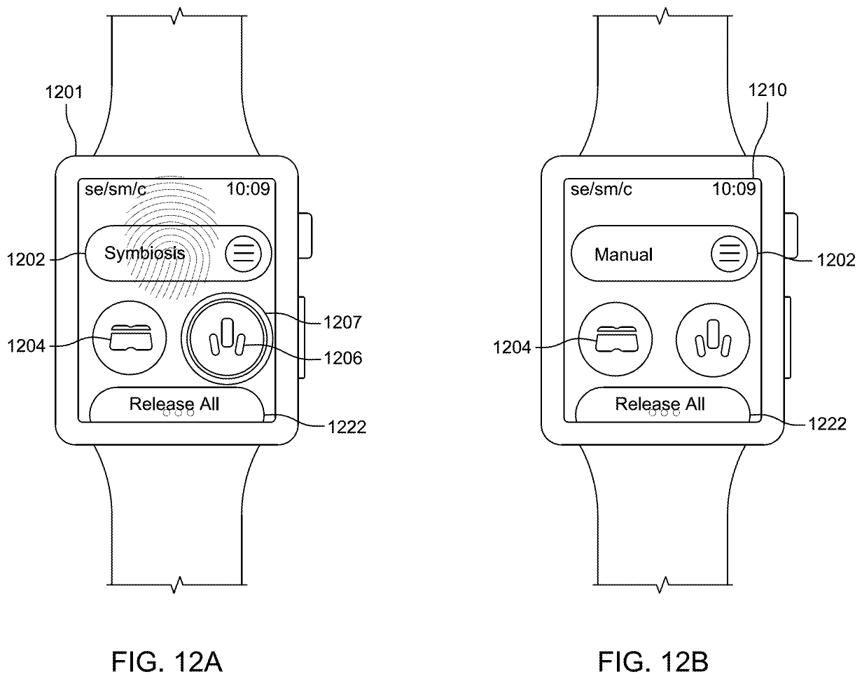

FIGS. 12A-12B show different screens showing symbiosis mode being turned OFF according to an embodiment. FIG. 12A shows screen 1201 having symbiosis ON/OFF button 1202, lumbar support button 1204, and standing support button 1206 with timer 1207. Screen 1201 shows that button 1202 is ON and that lumbar support assistance 1204 is ON, and that timer 1207 is active (as indicated by the presence of progress circle). If the user presses button 1202 to toggle it OFF, screen 1210 of FIG. 12B is shown. FIG. 12B shows that all timers are deactivated, and that lumbar support 1204 remains active. The user can manually turn off support by pressing release button 1222.

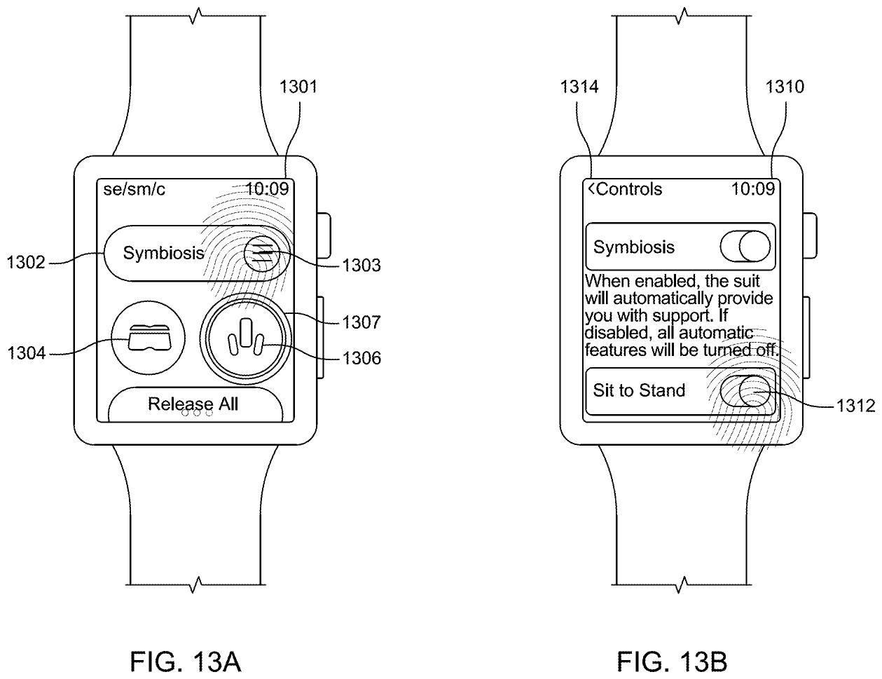

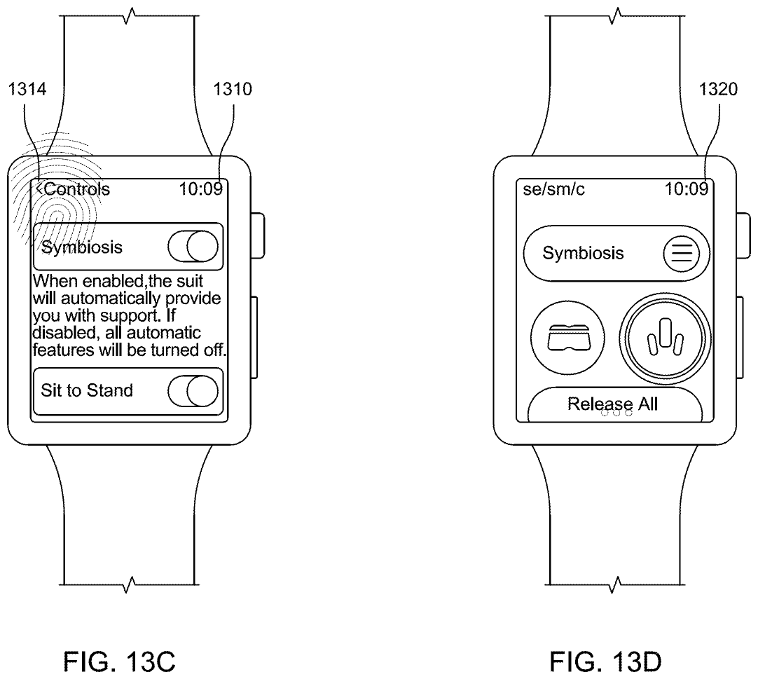

FIGS. 13A-13D show different screens related to sit-to-stand according to an embodiment. FIG. 13A shows screen 1301 that include symbiosis ON/OFF button 1302, symbiosis submenu button 1303, lumbar support button 1304, standing support button 1306 with timer 1307, and release button 1322. Screen 1301 indicates symbiosis mode is ON and the lumbar support 1304 is ON. If the user presses submenu button 1303, screen 1310 of FIG. 13B is shown. Screen 1300 shows a scrollable settings screen that enables the user to turn symbiosis ON/OFF, and the sit to stand assist feature ON/OFF. If the user turns sit to stand OFF by pressing button 1312, the sit-to-stand assist is turned off but the symbiosis timers for other assistance feature (e.g., lumbar support) remain on. The user can exit out of settings screen 1310 by pressing back button (shown in FIG. 13C) to return to screen 1320 of FIG. 13D.

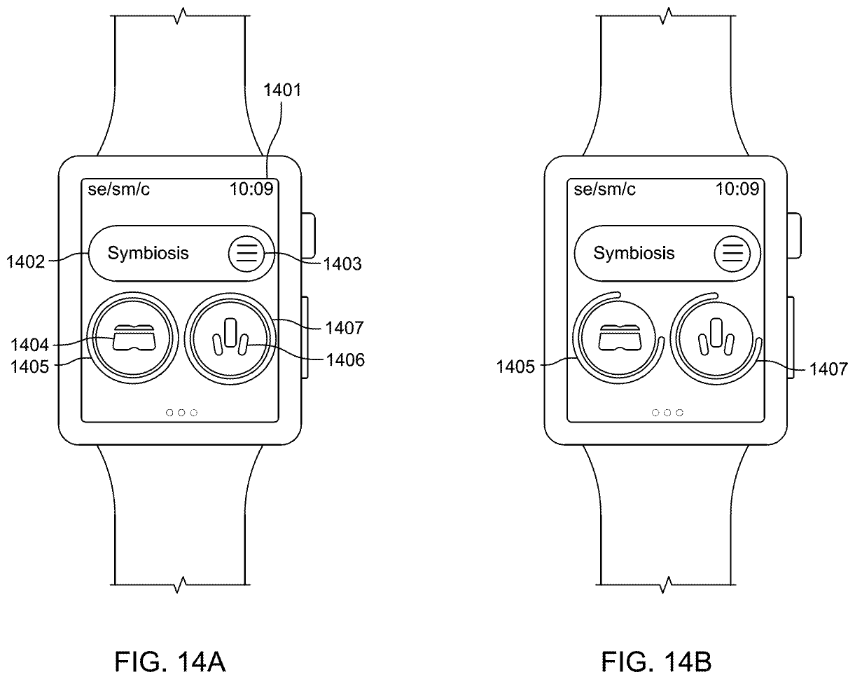

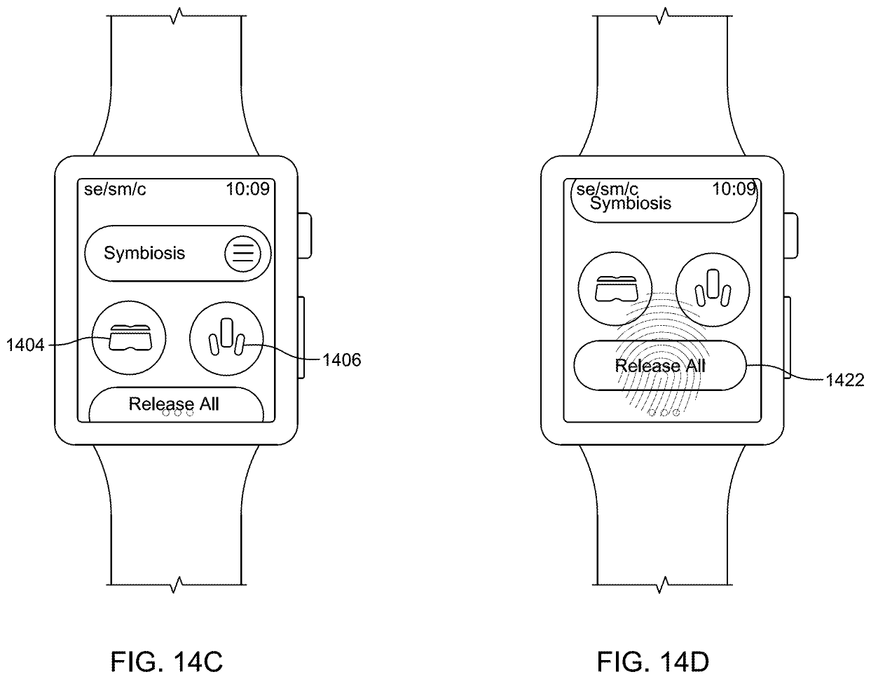

FIGS. 14A-14D show different screens that show standing support and lumbar support via timers according to an embodiment. FIG. 14A shows screen 1401 that include symbiosis ON/OFF button 1402, symbiosis submenu button 1403, lumbar support button 1404 with timer 1405, standing support button 1406 with timer 1407. Symbiosis button 1402 is ON, and the suit recognizes that the user is in the standing position. When the user is recognized as standing, both lumbar support and standing support assistance functions go into respective base tone modes to prepare for providing assistance. FIG. 14B shows that countdown timers 1405 and 1407 have commenced. Countdown timers 1405 and 1407 may continue to countdown provided the user does not walk, lean, shift, or sit down prior to countdown timer completion. FIG. 14C shows that both countdown timers have elapsed and both lumber support button 1404 and standing support button 1406 are ON. FIG. 14D shows that the user can press release button 1422 to cease all support actions.

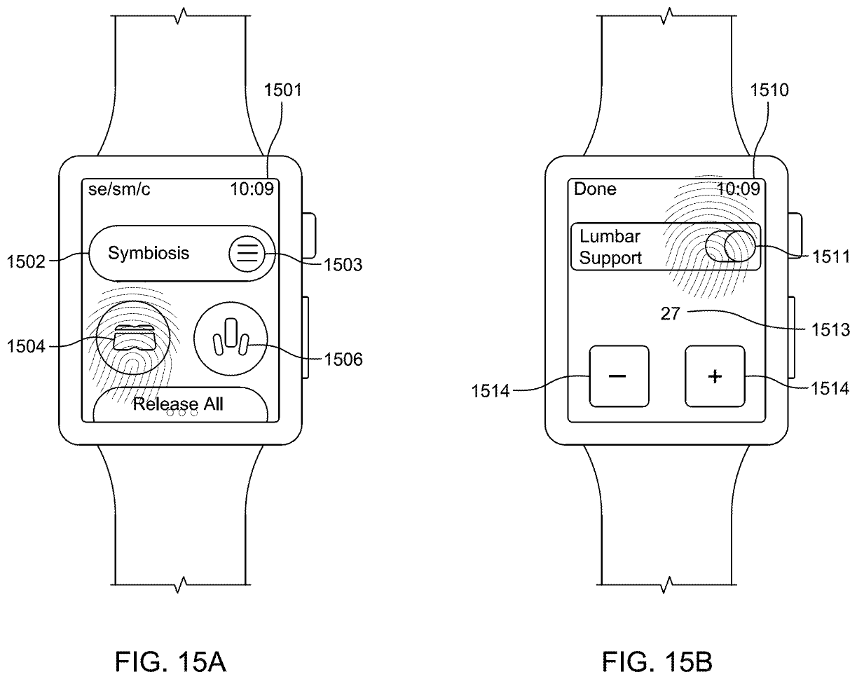

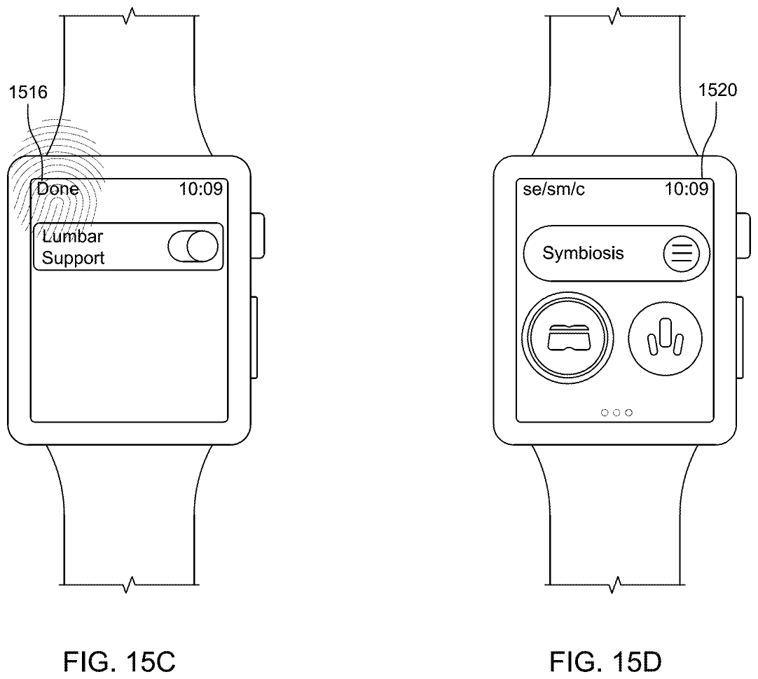

FIGS. 15A-15D show different screens related to lumbar support according to an embodiment. FIG. 15A shows screen 1501 that include symbiosis ON/OFF button 1502, symbiosis submenu button 1503, lumbar support button 1504, and standing support button 1506. Screen 1501 indicates lumbar support is ON and the standing support is OFF. When the user presses down on button 1504, screen 1510 of FIG. 15B can be displayed. Screen 1510 is a settings screen for lumbar support and shows ON/OFF button 1511, lumbar assistance status 1513 and down and up buttons 1514 and 1515. If the user turns lumbar support OFF as shown in FIG. 15C, status 1513, and down and up buttons 1514 and 1515 are removed. If the user presses done button 1516, screen 1520 of FIG. 15D can be displayed.

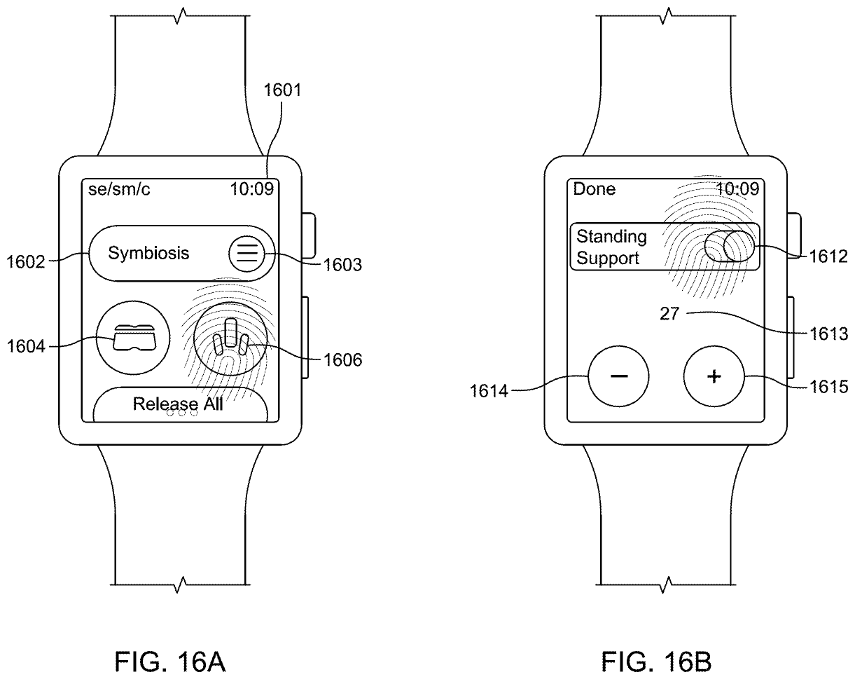

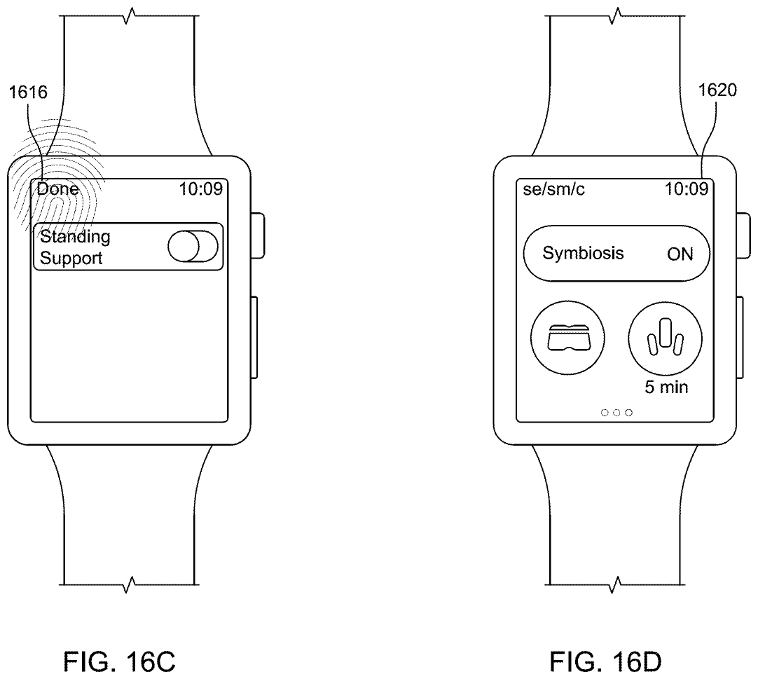

FIGS. 16A-16D show different screens related to standing support according to an embodiment. FIG. 16A shows screen 1601 that include symbiosis ON/OFF button 1602, symbiosis submenu button 1603, lumbar support button 1604, and standing support button 1606. Screen 1601 indicates lumbar support is ON and the standing support is ON. When the user presses down on button 1606, screen 1610 of FIG. 16B can be displayed. Screen 1610 is a settings screen for standing support and shows ON/OFF button 1611, standing assistance status 1613 and down and up buttons 1614 and 1615. If the user turns standing support OFF as shown in FIG. 16C, status 1613, and down and up buttons 1614 and 1615 are removed. If the user presses done button 1616, screen 1620 of FIG. 16D can be displayed.

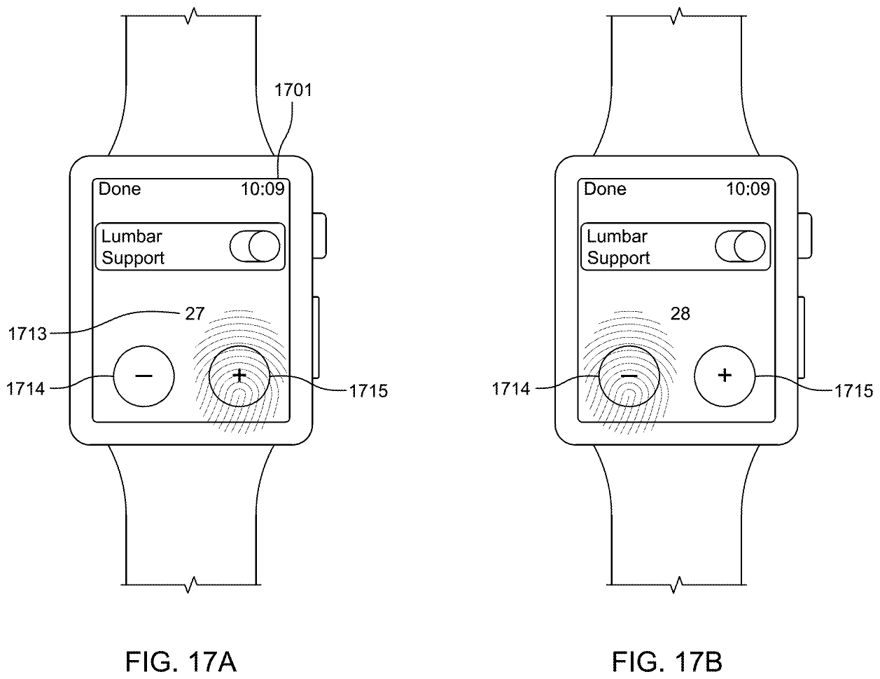

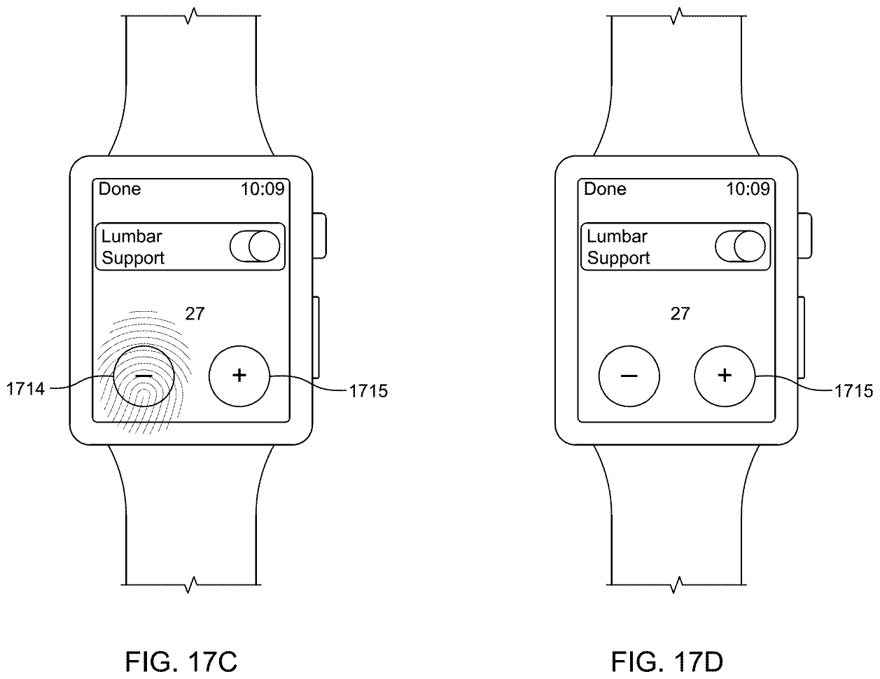

FIGS. 17A-17D show different screens related to adjusting lumbar support according to an embodiment. FIG. 17A-17D shows that the use can touch down and up buttons 1714 and 1715 to change the lumbar setting, as shown in status 1713. When the user presses up button 1715 (in FIG. 17A), status 1713 changes from 27 to 28, as shown in FIG. 17B. When the user presses down button 1714 (in FIG. 17B), status 1713 changes from 28 to 27, as shown in FIG. 17C. If lumbar setting cannot down any further, down button 1714 may be deactivated. Similarly, if the lumbar setting cannot go up any further, up button 1715 may be deactivated.

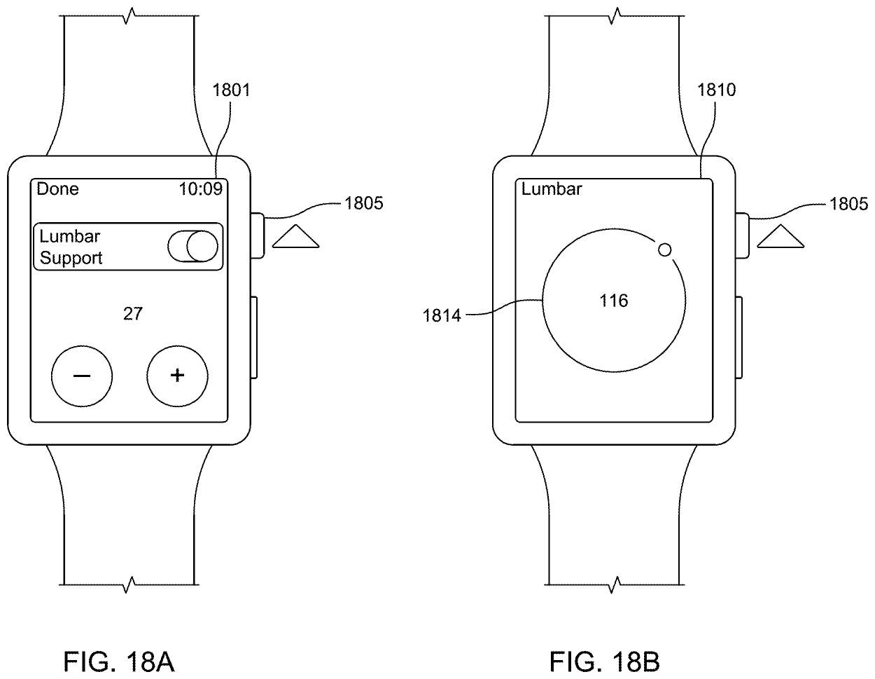

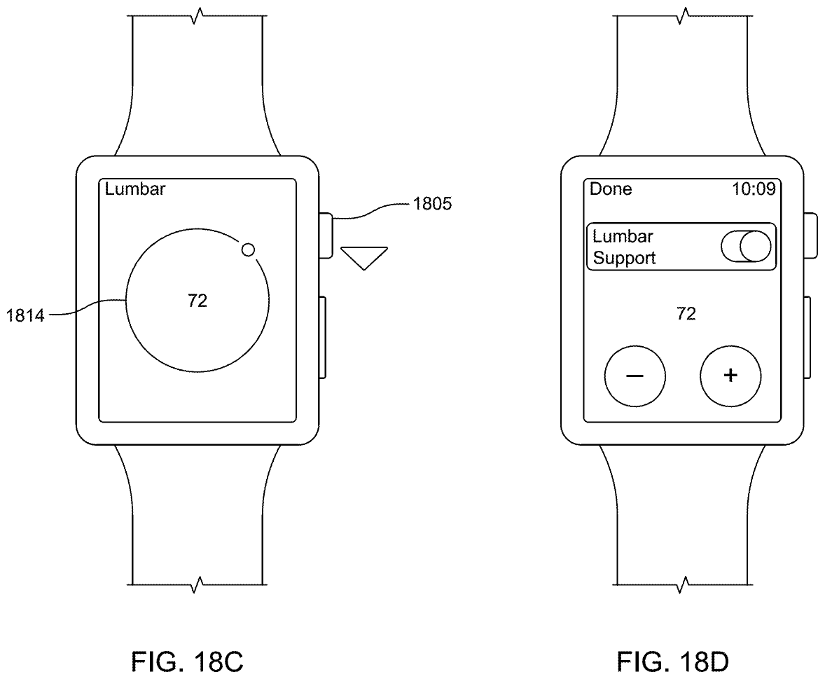

FIGS. 18A-18D show different screens related to adjusting lumbar support according to an embodiment. Screen 1801 shows a lumbar setting screen. If user interacts with rotatable crown input 1805, for example, by rotating it clockwise, the lumbar setting may be increased. This is shown in FIG. 18B which shows that the lumbar setting has been increased from 27 to 116. In addition, screen 1810 shows circular display 1814. In FIG. 18C, the user can rotate crown input 1805 counter clockwise to decrease lumbar force. When the user stops turning crown input 1805, the user may be presented with screen 1820 of FIG. 18D.

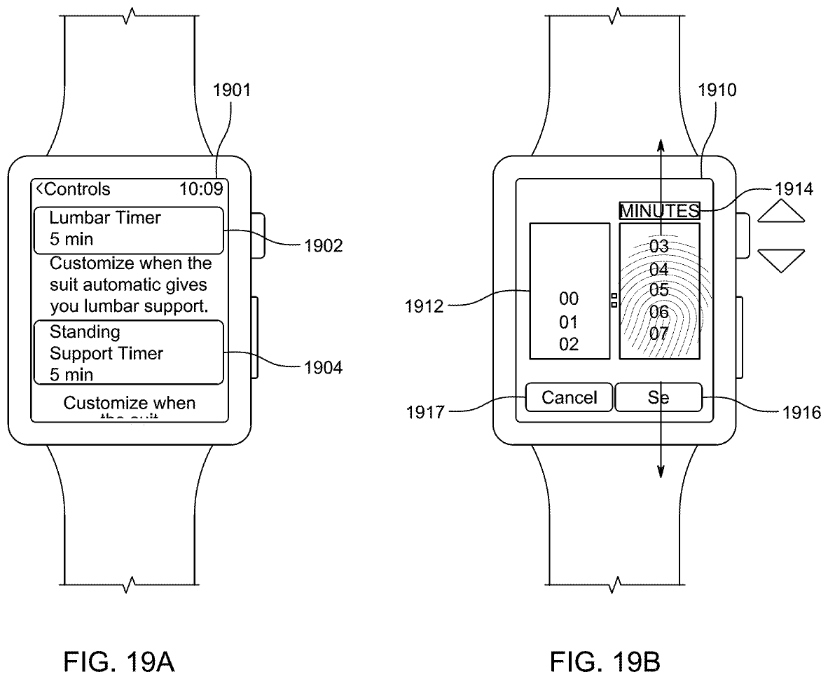

FIGS. 19A and 19B show how the timers for lumbar support and standing support can be set according to various embodiments. FIG. 19A shows a setting screen 1901 where the user can set timers for lumbar support at button 1902 and for standing support at button 1904. As shown, both timers are set for 5 minutes. This means that the suit will automatically provide lumbar and standing support if the user has been standing uninterrupted for five minutes. If the user selects one of buttons 1902 and 1904, screen 1910 of FIG. 19B can be displayed. Screen 1910 shows hour window 1912 and minute window 1914 that allows the user to set the timer length. When the user finished setting the timer duration, the user can press set button 1916 or can press cancel button 1917 to cancel the timer setting and return to screen 1901.

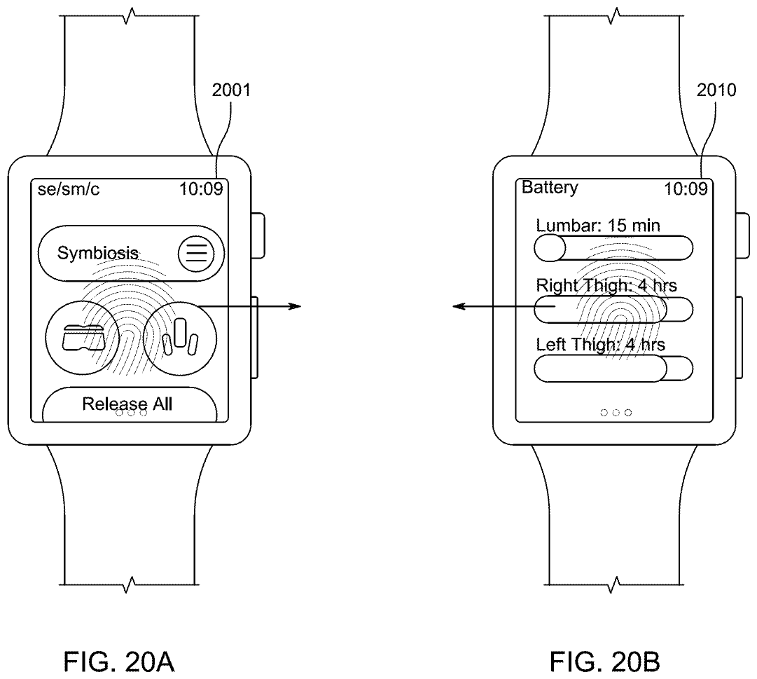

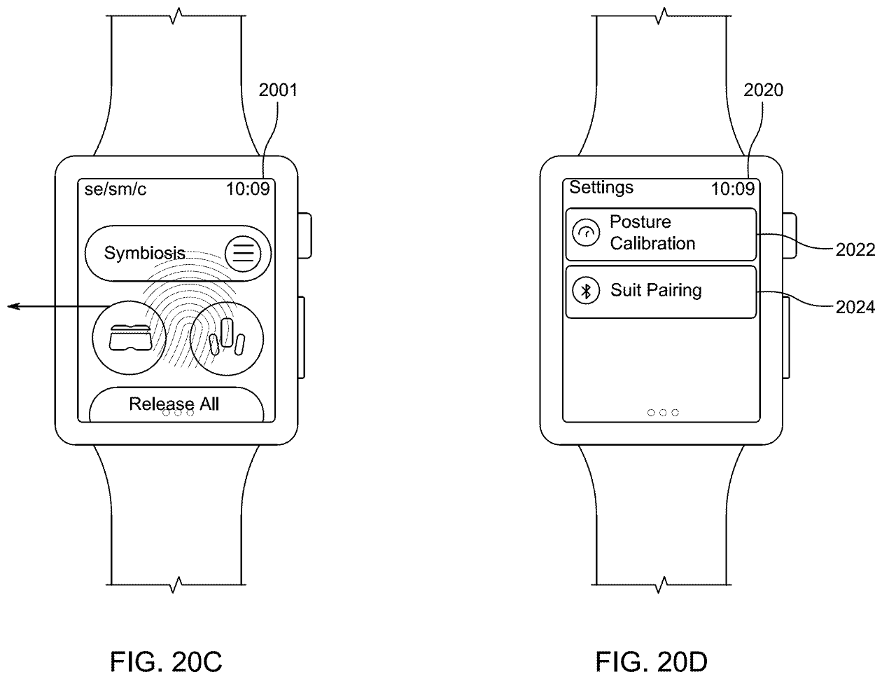

FIGS. 20A-20D show different screens that may be displayed according to various embodiments. FIG. 20A shows home screen 2001, and when the user swipes to the right, battery status screen 2010 is displayed. Screen 2010 shows the remaining power for several power layers (e.g., lumbar, right thigh, and left thigh). If the user swipes left, screen 2001 of FIG. 20C is shown, and if the user swipes again to the left, settings screen 2020 is shown in FIG. 20D. Screen 2020 includes posture calibration menu 2022 and suit pair menu 2024.

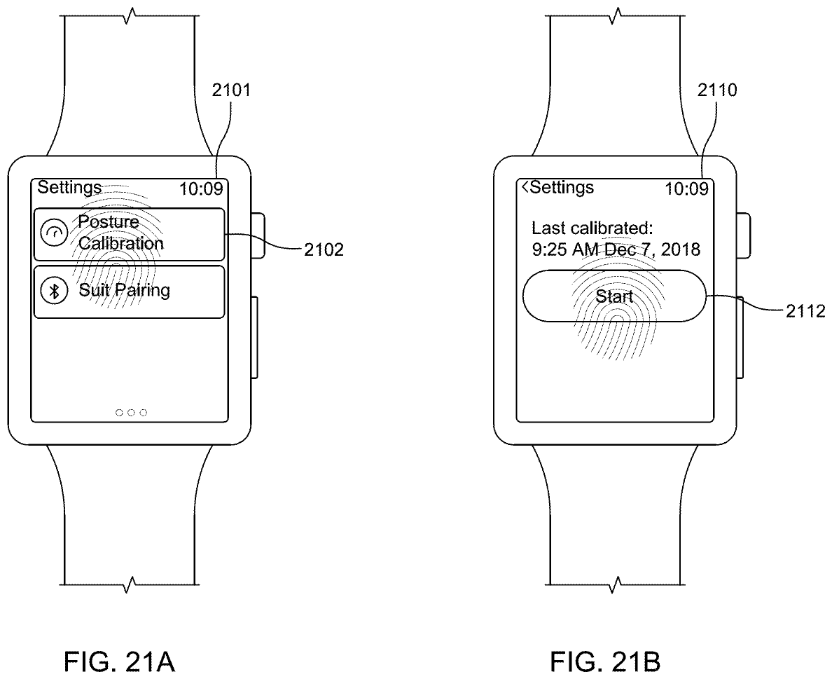

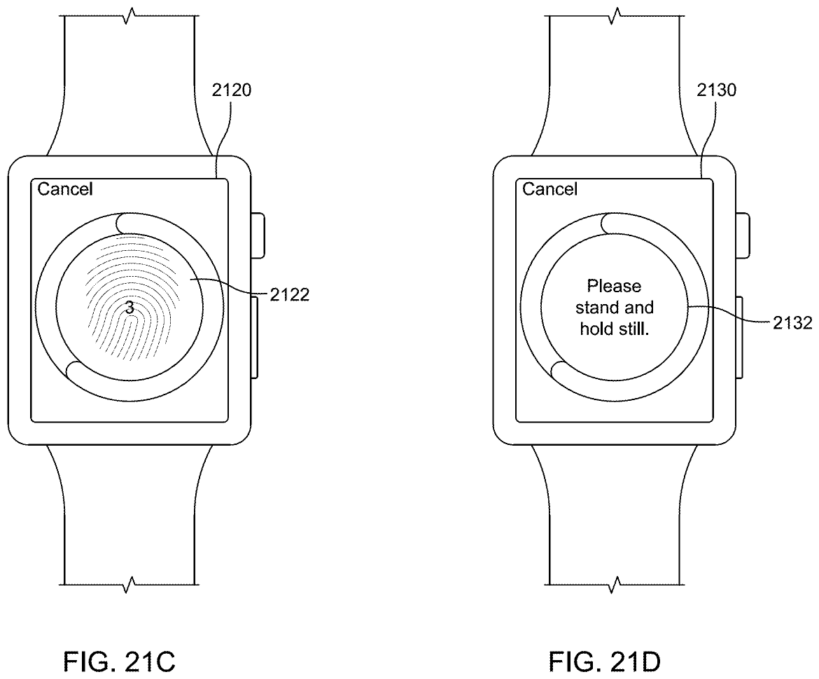

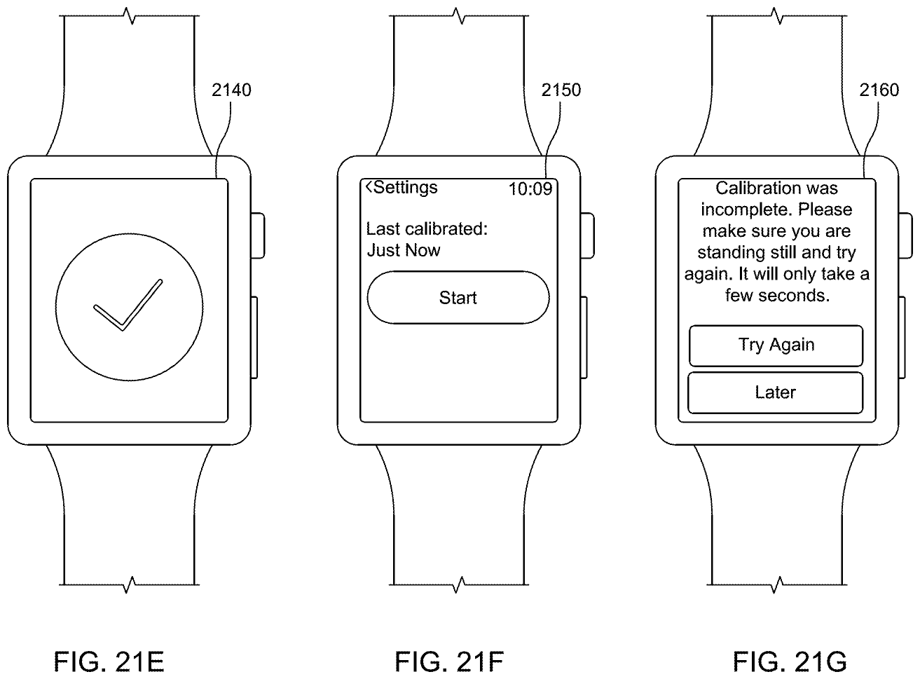

FIGS. 21A-21G show different calibration screens that may be displayed according to various embodiments. FIG. 21A shows a user selecting posture calibration menu 2102 in screen 2101. Screen 2120 shows the last time the suit calibrated (if it was calibrated) and had start button 2122. When start button 2122 is pressed, count down timer 2122 of screen 2120 is shown in FIG. 21C. Info screen 2132 informs the user to stand and hold still in screen 2130 after the countdown timer has elapsed. The suit may calibrate itself while info screen 2132 is shown. Screen 2140 may be displayed to show that the calibration is complete. Screen 2150 may show the updated calibration time. If the calibration failed, screen 216 of FIG. 21G may be displayed.

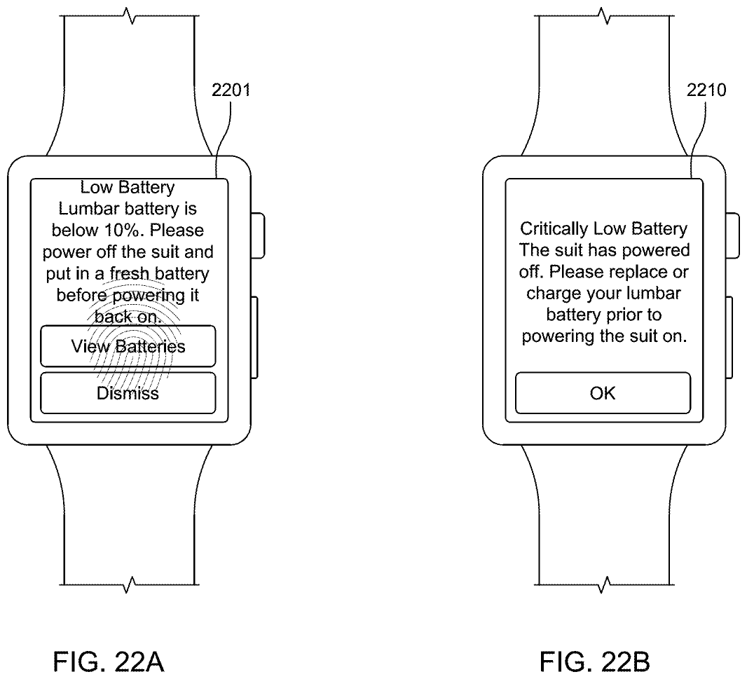

FIGS. 22A and 22B show different battery status screens that may be displayed according to various embodiments. FIG. 22A shows warning screen 2201 that indicates that a particular battery's power level is low. FIG. 22B shows a warning screen 2210 that indicates that the battery for a particular power layer has dropped below a threshold and that the suit has powered off.

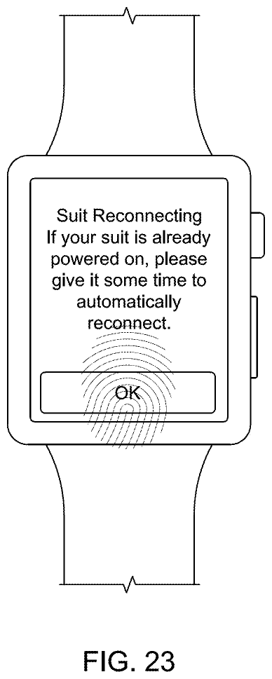

FIG. 23 shows a warning screen 2300 indicating the personal device lost connection with the exosuit.

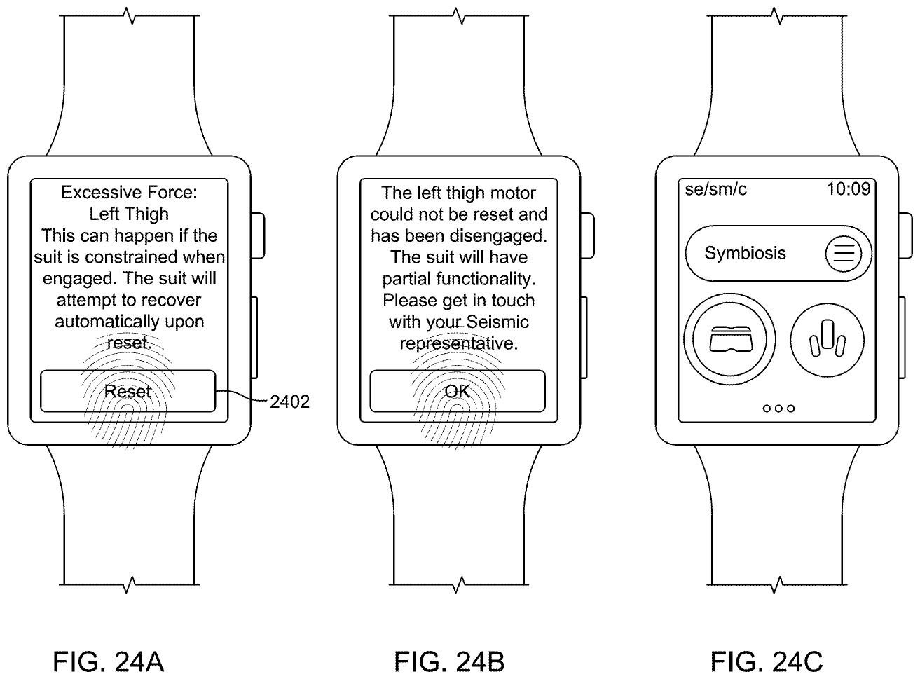

FIGS. 24A-24C show alert screens that may be displayed according to various embodiments. FIG. 24A shows that a condition has been detected with a particular power layer (e.g., left thigh) and asks the user to reset it via button 2402. If the power layer cannot be reset, the user may be presented with the screen in FIG. 24B. FIG. 24C demonstrates that when certain assist features are disabled, such a feature will be grayed out or visually indicated that such feature no longer functions.

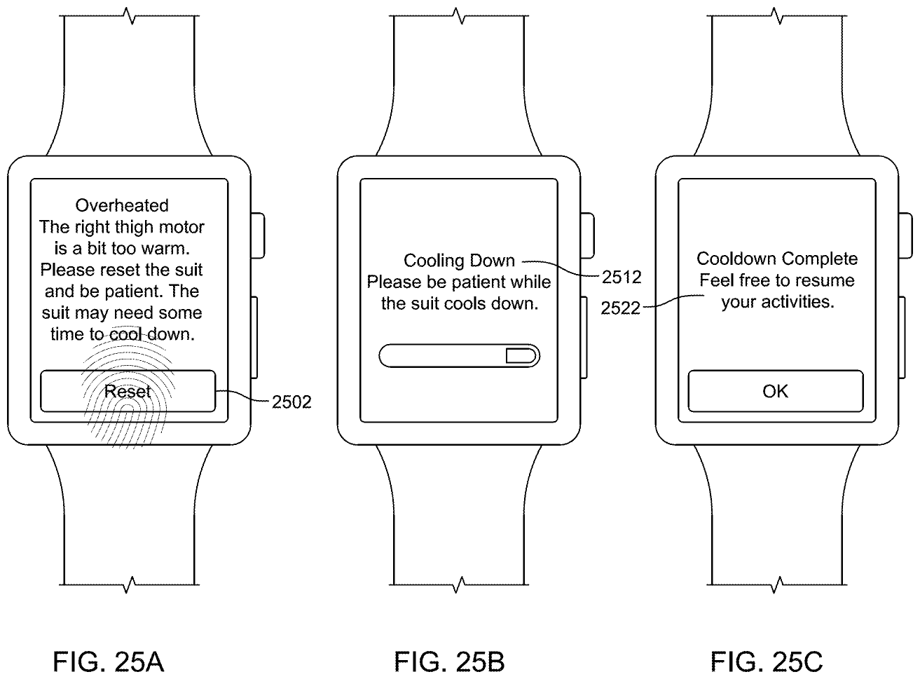

FIGS. 25A-25C show high heat alert screens that may be displayed according to various embodiments. FIG. 25A shows a warning screen indicating that a power layer has overheated and that it should be reset via button 2502. If reset is successful, cool down progress screen, such as screen 2512 can be displayed as shown in FIG. 25B. If the cool down is successful, screen 2522 can be displayed as shown in FIG. 25C.

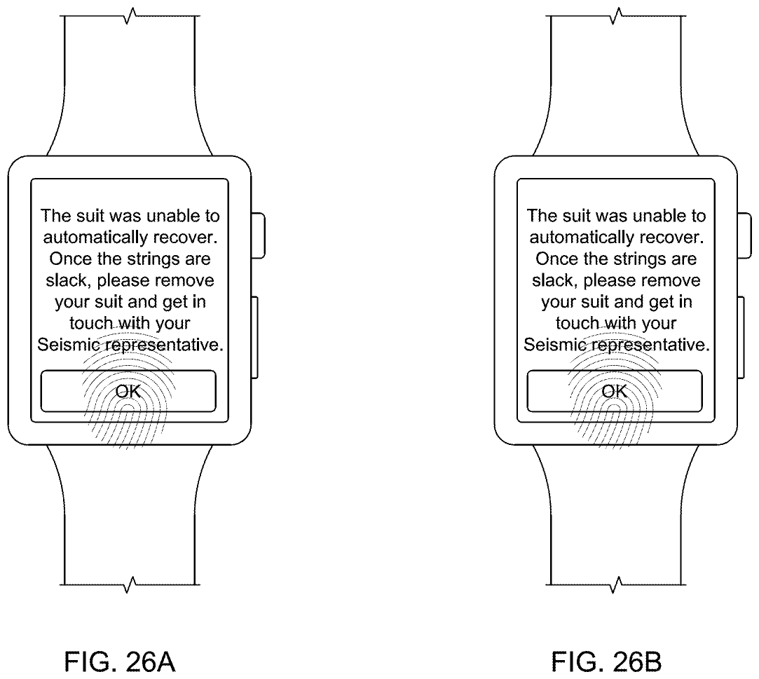

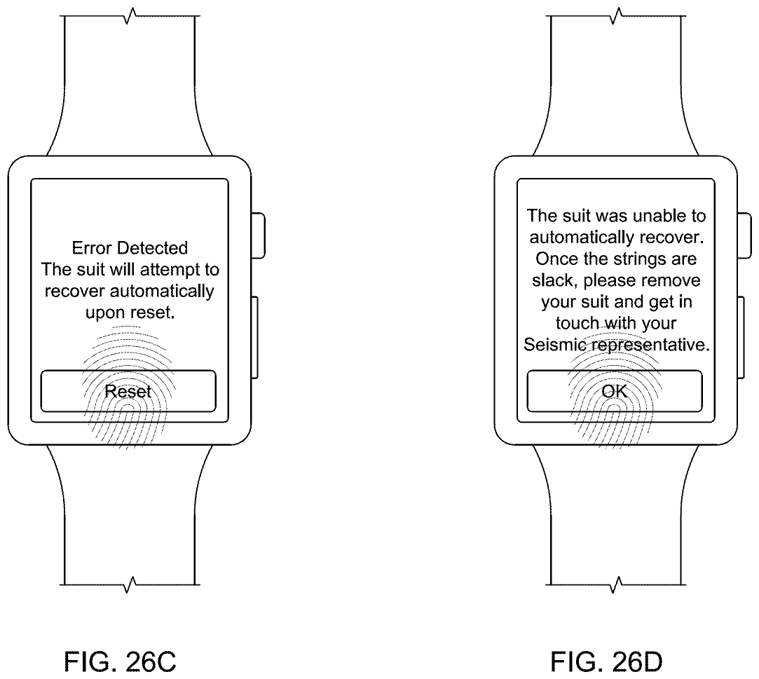

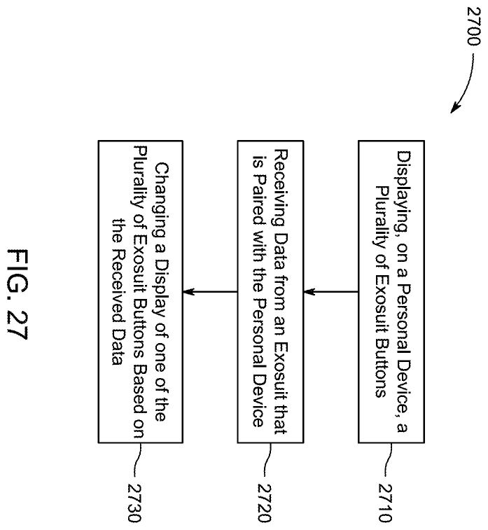

FIGS. 26A-26D show other alert screens that may be displayed according to various embodiments.

FIG. 27 shows an illustrative process 2700 according to an embodiment. Process 2700 may begin at step 2710 by displaying, on a personal device, a plurality of exosuit buttons. For example, any one the screens in FIGS. 9-26 can be displayed. At step 2720, data can be received from an exosuit that is paired with the personal device. For example, if the exosuit had detected that the user is standing, it may communicate that data to personal device. At step 2730, a display of one of the plurality of exosuit buttons can be changed based on the received data. For example, if exosuit confirms the use is standing, the countdown timer for one or more assistance movement may be displayed.

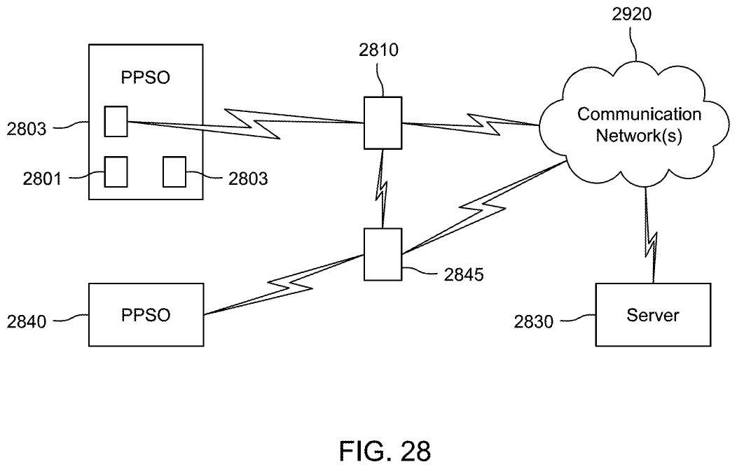

FIG. 28 illustrates an example exosuit 2800 that includes actuators 2801, sensors 2803, and a controller configured to operate elements of exosuit 2800 (e.g., 2801, 2803) to enable functions of the exosuit 2800. The controller 2805 is configured to communicate wirelessly with a user interface 2810. The user interface 2810 is configured to present information to a user (e.g., a wearer of the exosuit 2800) and to the controller 2805 of the flexible exosuit or to other systems. The user interface 2810 can be involved in controlling and/or accessing information from elements of the exosuit 2800. For example, an application being executed by the user interface 2810 can access data from the sensors 2803, calculate an operation (e.g., to apply dorsiflexion stretch) of the actuators 2801, and transmit the calculated operation to the exosuit 2800. The user interface 2810 can additionally be configured to enable other functions; for example, the user interface 2810 can be configured to be used as a cellular telephone, a portable computer, an entertainment device, or to operate according to other applications.

The user interface 2810 can be configured to be removably mounted to the exosuit 2800 (e.g., by straps, magnets, Velcro, charging and/or data cables). Alternatively, the user interface 2810 can be configured as a part of the exosuit 2800 and not to be removed during normal operation. In some examples, a user interface can be incorporated as part of the exosuit 2800 (e.g., a touchscreen integrated into a sleeve of the exosuit 2800) and can be used to control and/or access information about the exosuit 2800 in addition to using the user interface 2810 to control and/or access information about the exosuit 2800. In some examples, the controller 2805 or other elements of the exosuit 2800 are configured to enable wireless or wired communication according to a standard protocol (e.g., Bluetooth, ZigBee, WiFi, LTE or other cellular standards, IRdA, Ethernet) such that a variety of systems and devices can be made to operate as the user interface 2810 when configured with complementary communications elements and computer-readable programs to enable such functionality.

The exosuit 2800 can be configured as described in example embodiments herein or in other ways according to an application. The exosuit 2800 can be operated to enable a variety of applications. The exosuit 2800 can be operated to enhance the strength of a wearer by detecting motions of the wearer (e.g., using sensors 2803) and responsively applying torques and/or forces to the body of the wearer (e.g., using actuators 2801) to increase the forces the wearer is able to apply to his/her body and/or environment. The exosuit 2800 can be operated to train a wearer to perform certain physical activities. For example, the exosuit 2800 can be operated to enable rehabilitative therapy of a wearer. The exosuit 2800 can operate to amplify motions and/or forces produced by a wearer undergoing therapy in order to enable the wearer to successfully complete a program of rehabilitative therapy. Additionally or alternatively, the exosuit 2800 can be operated to prohibit disordered movements of the wearer and/or to use the actuators 2801 and/or other elements (e.g., haptic feedback elements) to indicate to the wearer a motion or action to perform and/or motions or actions that should not be performed or that should be terminated. Similarly, other programs of physical training (e.g., dancing, skating, other athletic activities, vocational training) can be enabled by operation of the exosuit 2800 to detect motions, torques, or forces generated by a wearer and/or to apply forces, torques, or other haptic feedback to the wearer. Other applications of the exosuit 2800 and/or user interface 2810 are anticipated.

The user interface 2810 can additionally communicate with communications network(s) 2820. For example, the user interface 2810 can include a WiFi radio, an LTE transceiver or other cellular communications equipment, a wired modem, or some other elements to enable the user interface 2810 and exosuit 2800 to communicate with the Internet. The user interface 2810 can communicate through the communications network 2820 with a server 2830. Communication with the server 2830 can enable functions of the user interface 2810 and exosuit 2800. In some examples, the user interface 2810 can upload telemetry data (e.g., location, configuration of elements 2801, 2803 of the exosuit 2800, physiological data about a wearer of the exosuit 2800) to the server 2830.

In some examples, the server 2830 can be configured to control and/or access information from elements of the exosuit 2800 (e.g., 2801, 2803) to enable some application of the exosuit 2800. For example, the server 2830 can operate elements of the exosuit 2800 to move a wearer out of a dangerous situation if the wearer was injured, unconscious, or otherwise unable to move themselves and/or operate the exosuit 2800 and user interface 2810 to move themselves out of the dangerous situation. Other applications of a server in communications with a exosuit are anticipated.

The user interface 2810 can be configured to communicate with a second user interface 2845 in communication with and configured to operate a second flexible exosuit 2840. Such communication can be direct (e.g., using radio transceivers or other elements to transmit and receive information over a direct wireless or wired link between the user interface 2810 and the second user interface 2845). Additionally or alternatively, communication between the user interface 2810 and the second user interface 2845 can be facilitated by communications network(s) 2820 and/or a server 2830 configured to communicate with the user interface 2810 and the second user interface 2845 through the communications network(s) 2820.

Communication between the user interface 2810 and the second user interface 2845 can enable applications of the exosuit 2800 and second exosuit 2840. In some examples, actions of the exosuit 2800 and second flexible exosuit 2840 and/or of wearers of the exosuit 2800 and second exosuit 2840 can be coordinated. For example, the exosuit 2800 and second exosuit 2840 can be operated to coordinate the lifting of a heavy object by the wearers. The timing of the lift, and the degree of support provided by each of the wearers and/or the exosuit 2800 and second exosuit 2840 can be controlled to increase the stability with which the heavy object was carried, to reduce the risk of injury of the wearers, or according to some other consideration. Coordination of actions of the exosuit 2800 and second exosuit 2840 and/or of wearers thereof can include applying coordinated (in time, amplitude, or other properties) forces and/or torques to the wearers and/or elements of the environment of the wearers and/or applying haptic feedback (though actuators of the exosuits 2800, 2840, through dedicated haptic feedback elements, or through other methods) to the wearers to guide the wearers toward acting in a coordinated manner.

Coordinated operation of the exosuit 2800 and second exosuit 2840 can be implemented in a variety of ways. In some examples, one exosuit (and the wearer thereof) can act as a master, providing commands or other information to the other exosuit such that operations of the exosuit 2800, 2840 are coordinated. For example, the exosuit 2800, 2840 can be operated to enable the wearers to dance (or to engage in some other athletic activity) in a coordinated manner. One of the exosuits can act as the ‘lead’, transmitting timing or other information about the actions performed by the ‘lead’ wearer to the other exosuit, enabling coordinated dancing motions to be executed by the other wearer. In some examples, a first wearer of a first exosuit can act as a trainer, modeling motions or other physical activities that a second wearer of a second exosuit can learn to perform. The first exosuit can detect motions, torques, forces, or other physical activities executed by the first wearer and can send information related to the detected activities to the second exosuit. The second exosuit can then apply forces, torques, haptic feedback, or other information to the body of the second wearer to enable the second wearer to learn the motions or other physical activities modeled by the first wearer. In some examples, the server 2830 can send commands or other information to the exosuits 2800, 2840 to enable coordinated operation of the exosuits 2800, 2840.

The exosuit 2800 can be operated to transmit and/or record information about the actions of a wearer, the environment of the wearer, or other information about a wearer of the exosuit 2800. In some examples, kinematics related to motions and actions of the wearer can be recorded and/or sent to the server 2830. These data can be collected for medical, scientific, entertainment, social media, or other applications. The data can be used to operate a system. For example, the exosuit 2800 can be configured to transmit motions, forces, and/or torques generated by a user to a robotic system (e.g., a robotic arm, leg, torso, humanoid body, or some other robotic system) and the robotic system can be configured to mimic the activity of the wearer and/or to map the activity of the wearer into motions, forces, or torques of elements of the robotic system. In another example, the data can be used to operate a virtual avatar of the wearer, such that the motions of the avatar mirrored or were somehow related to the motions of the wearer. The virtual avatar can be instantiated in a virtual environment, presented to an individual or system with which the wearer is communicating, or configured and operated according to some other application.

Conversely, the exosuit 2800 can be operated to present haptic or other data to the wearer. In some examples, the actuators 2801 (e.g., twisted string actuators, exotendons) and/or haptic feedback elements (e.g., EPAM haptic elements) can be operated to apply and/or modulate forces applied to the body of the wearer to indicate mechanical or other information to the wearer. For example, the activation in a certain pattern of a haptic element of the exosuit 2800 disposed in a certain location of the exosuit 2800 can indicate that the wearer had received a call, email, or other communications. In another example, a robotic system can be operated using motions, forces, and/or torques generated by the wearer and transmitted to the robotic system by the exosuit 2800. Forces, moments, and other aspects of the environment and operation of the robotic system can be transmitted to the exosuit 2800 and presented (using actuators 2801 or other haptic feedback elements) to the wearer to enable the wearer to experience force-feedback or other haptic sensations related to the wearer's operation of the robotic system. In another example, haptic data presented to a wearer can be generated by a virtual environment, e.g., an environment containing an avatar of the wearer that is being operated based on motions or other data related to the wearer that is being detected by the exosuit 2800.

Note that the exosuit 2800 illustrated in FIG. 28 is only one example of a exosuit that can be operated by control electronics, software, or algorithms described herein. Control electronics, software, or algorithms as described herein can be configured to control flexible exosuits or other mechatronic and/or robotic system having more, fewer, or different actuators, sensors or other elements. Further, control electronics, software, or algorithms as described herein can be configured to control exosuits configured similarly to or differently from the illustrated exosuit 2800. Further, control electronics, software, or algorithms as described herein can be configured to control flexible exosuits having reconfigurable hardware (i.e., exosuits that are able to have actuators, sensors, or other elements added or removed) and/or to detect a current hardware configuration of the flexible exosuits using a variety of methods.

A controller of a exosuit and/or computer-readable programs executed by the controller can be configured to provide encapsulation of functions and/or components of the flexible exosuit. That is, some elements of the controller (e.g., subroutines, drivers, services, daemons, functions) can be configured to operate specific elements of the exosuit (e.g., a twisted string actuator, a haptic feedback element) and to allow other elements of the controller (e.g., other programs) to operate the specific elements and/or to provide abstracted access to the specific elements (e.g., to translate a command to orient an actuator in a commanded direction into a set of commands sufficient to orient the actuator in the commanded direction). This encapsulation can allow a variety of services, drivers, daemons, or other computer-readable programs to be developed for a variety of applications of a flexible exosuits. Further, by providing encapsulation of functions of a flexible exosuit in a generic, accessible manner (e.g., by specifying and implementing an application programming interface (API) or other interface standard), computer-readable programs can be created to interface with the generic, encapsulated functions such that the computer-readable programs can enable operating modes or functions for a variety of differently-configured exosuit, rather than for a single type or model of flexible exosuit. For example, a virtual avatar communications program can access information about the posture of a wearer of a flexible exosuit by accessing a standard exosuit API. Differently-configured exosuits can include different sensors, actuators, and other elements, but can provide posture information in the same format according to the API. Other functions and features of a flexible exosuit, or other robotic, exoskeletal, assistive, haptic, or other mechatronic system, can be encapsulated by APIs or according to some other standardized computer access and control interface scheme.

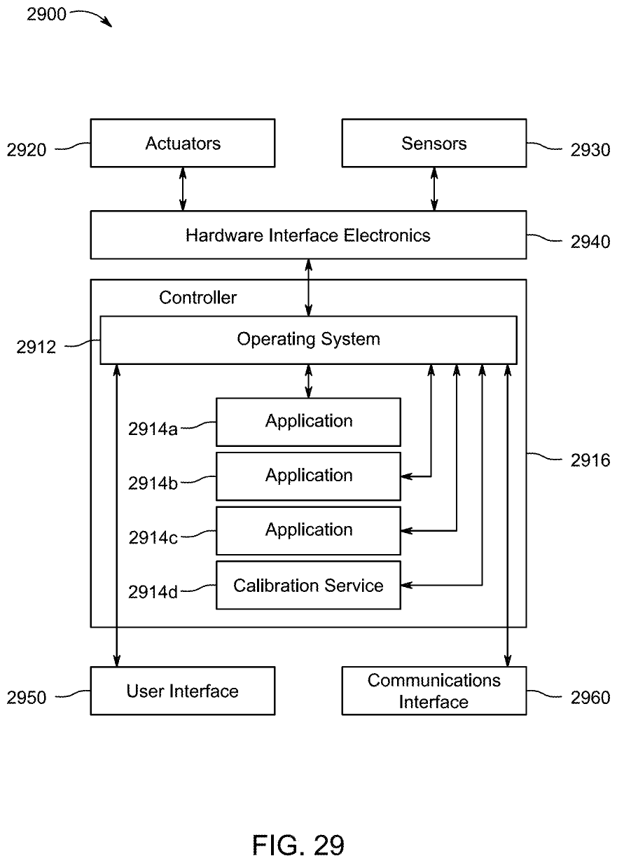

FIG. 29 is a schematic illustrating elements of a exosuit 2900 and a hierarchy of control or operating the exosuit 2900. The flexible exosuit includes actuators 2920 and sensors 2930 configured to apply forces and/or torques to and detect one or more properties of, respectively, the exosuit 2900, a wearer of the exosuit 2900, and/or the environment of the wearer. The exosuit 2900 additionally includes a controller 2910 configured to operate the actuators 2920 and sensors 2930 by using hardware interface electronics 2940. The hardware electronics interface 2940 includes electronics configured to interface signals from and to the controller 2910 with signals used to operate the actuators 2920 and sensors 2930. For example, the actuators 2920 can include exotendons, and the hardware interface electronics 2940 can include high-voltage generators, high-voltage switches, and high-voltage capacitance meters to clutch and un-clutch the exotendons and to report the length of the exotendons. The hardware interface electronics 2940 can include voltage regulators, high voltage generators, amplifiers, current detectors, encoders, magnetometers, switches, controlled-current sources, DACs, ADCs, feedback controllers, brushless motor controllers, or other electronic and mechatronic elements.

The controller 2910 additionally operates a user interface 2950 that is configured to present information to a user and/or wearer of the exosuit 2900 and a communications interface 2960 that is configured to facilitate the transfer of information between the controller 2910 and some other system (e.g., by transmitting a wireless signal). Additionally or alternatively, the user interface 2950 can be part of a separate system that is configured to transmit and receive user interface information to/from the controller 2910 using the communications interface 2960 (e.g., the user interface 2950 can be part of a cellphone).

The controller 2910 is configured to execute computer-readable programs describing functions of the flexible exosuit 2912. Among the computer-readable programs executed by the controller 2910 are an operating system 2912, applications 2914 a, 2914 b, 2914 c, and a calibration service 2916. The operating system 2912 manages hardware resources of the controller 2910 (e.g., I/O ports, registers, timers, interrupts, peripherals, memory management units, serial and/or parallel communications units) and, by extension, manages the hardware resources of the exosuit 2900. The operating system 2912 is the only computer-readable program executed by the controller 2910 that has direct access to the hardware interface electronics 2940 and, by extension, the actuators 2920 and sensors 2930 of the exosuit 2900.

The applications 2914 a, 2914 b, 2914 are computer-readable programs that describe some function, functions, operating mode, or operating modes of the exosuit 2900. For example, application 2914 a can describe a process for transmitting information about the wearer's posture to update a virtual avatar of the wearer that includes accessing information on a wearer's posture from the operating system 2912, maintaining communications with a remote system using the communications interface 2960, formatting the posture information, and sending the posture information to the remote system. The calibration service 2916 is a computer-readable program describing processes to store parameters describing properties of wearers, actuators 2920, and/or sensors 2930 of the exosuit 2900, to update those parameters based on operation of the actuators 2920, and/or sensors 2930 when a wearer is using the exosuit 2900, to make the parameters available to the operating system 2912 and/or applications 2914 a, 2914 b, 2914 c, and other functions relating to the parameters. Note that applications 2914 a, 2914 b, 2914 and calibration service 2916 are intended as examples of computer-readable programs that can be run by the operating system 2912 of the controller 2910 to enable functions or operating modes of a exosuit 2900.

The operating system 2912 can provide for low-level control and maintenance of the hardware (e.g., 2920, 2930, 2940). In some examples, the operating system 2912 and/or hardware interface electronics 1540 can detect information about the exosuit 2900, the wearer, and/or the wearer's environment from one or more sensors 2930 at a constant specified rate. The operating system 2912 can generate an estimate of one or more states or properties of the exosuit 2900 or components thereof using the detected information. The operating system 2912 can update the generated estimate at the same rate as the constant specified rate or at a lower rate. The generated estimate can be generated from the detected information using a filter to remove noise, generate an estimate of an indirectly-detected property, or according to some other application. For example, the operating system 2912 can generate the estimate from the detected information using a Kalman filter to remove noise and to generate an estimate of a single directly or indirectly measured property of the exosuit 2900, the wearer, and/or the wearer's environment using more than one sensor. In some examples, the operating system can determine information about the wearer and/or exosuit 2900 based on detected information from multiple points in time. For example, the operating system 2900 can determine an eversion stretch and dorsiflexion stretch.

In some examples, the operating system 2912 and/or hardware interface electronics 2940 can operate and/or provide services related to operation of the actuators 2920. That is, in case where operation of the actuators 2920 requires the generation of control signals over a period of time, knowledge about a state or states of the actuators 2920, or other considerations, the operating system 2912 and/or hardware interface electronics 2940 can translate simple commands to operate the actuators 2920 (e.g., a command to generate a specified level of force using a twisted string actuator (TSA) of the actuators 2920) into the complex and/or state-based commands to the hardware interface electronics 2940 and/or actuators 2920 necessary to effect the simple command (e.g., a sequence of currents applied to windings of a motor of a TSA, based on a starting position of a rotor determined and stored by the operating system 2910, a relative position of the motor detected using an encoder, and a force generated by the TSA detected using a load cell).

In some examples, the operating system 2912 can further encapsulate the operation of the exosuit 2900 by translating a system-level simple command (e.g., a commanded level of force tension applied to the footplate) into commands for multiple actuators, according to the configuration of the exosuit 2900. This encapsulation can enable the creation of general-purpose applications that can effect a function of an exosuit (e.g., allowing a wearer of the exosuit to stretch his foot) without being configured to operate a specific model or type of exosuit (e.g., by being configured to generate a simple force production profile that the operating system 2912 and hardware interface electronics 2940 can translate into actuator commands sufficient to cause the actuators 2920 to apply the commanded force production profile to the footplate).

The operating system 2912 can act as a standard, multi-purpose platform to enable the use of a variety of exosuits having a variety of different hardware configurations to enable a variety of mechatronic, biomedical, human interface, training, rehabilitative, communications, and other applications. The operating system 2912 can make sensors 2930, actuators 2920, or other elements or functions of the exosuit 2900 available to remote systems in communication with the exosuit 2900 (e.g., using the communications interface 2960) and/or a variety of applications, daemons, services, or other computer-readable programs being executed by operating system 2912. The operating system 2912 can make the actuators, sensors, or other elements or functions available in a standard way (e.g., through an API, communications protocol, or other programmatic interface) such that applications, daemons, services, or other computer-readable programs can be created to be installed on, executed by, and operated to enable functions or operating modes of a variety of flexible exosuits having a variety of different configurations. The API, communications protocol, or other programmatic interface made available by the operating system 2912 can encapsulate, translate, or otherwise abstract the operation of the exosuit 2900 to enable the creation of such computer-readable programs that are able to operate to enable functions of a wide variety of differently-configured flexible exosuits.

Additionally or alternatively, the operating system 2912 can be configured to operate a modular flexible exosuit system (i.e., a flexible exosuit system wherein actuators, sensors, or other elements can be added or subtracted from a flexible exosuit to enable operating modes or functions of the flexible exosuit). In some examples, the operating system 2912 can determine the hardware configuration of the exosuit 2900 dynamically and can adjust the operation of the exosuit 2900 relative to the determined current hardware configuration of the exosuit 2900. This operation can be performed in a way that was ‘invisible’ to computer-readable programs (e.g., 2914 a, 2914 b, 2914 c) accessing the functionality of the exosuit 2900 through a standardized programmatic interface presented by the operating system 2912. For example, the computer-readable program can indicate to the operating system 2912, through the standardized programmatic interface, that a specified level of torque was to be applied to an ankle of a wearer of the exosuit 2900. The operating system 2912 can responsively determine a pattern of operation of the actuators 2920, based on the determined hardware configuration of the exosuit 2900, sufficient to apply the specified level of torque to the ankle of the wearer.

In some examples, the operating system 2912 and/or hardware interface electronics 2940 can operate the actuators 2920 to ensure that the exosuit 2900 does not operate to directly cause the wearer to be injured and/or elements of the exosuit 2900 to be damaged. In some examples, this can include not operating the actuators 2920 to apply forces and/or torques to the body of the wearer that exceeded some maximum threshold. This can be implemented as a watchdog process or some other computer-readable program that can be configured (when executed by the controller 2910) to monitor the forces being applied by the actuators 2920 (e.g., by monitoring commands sent to the actuators 2920 and/or monitoring measurements of forces or other properties detected using the sensors 2930) and to disable and/or change the operation of the actuators 2920 to prevent injury of the wearer. Additionally or alternatively, the hardware interface electronics 2940 can be configured to include circuitry to prevent excessive forces and/or torques from being applied to the wearer (e.g., by channeling to a comparator the output of a load cell that is configured to measure the force generated by a TSA, and configuring the comparator to cut the power to the motor of the TSA when the force exceeded a specified level).

In some examples, operating the actuators 2920 to ensure that the exosuit 2900 does not damage itself can include a watchdog process or circuitry configured to prevent over-current, over-load, over-rotation, or other conditions from occurring that can result in damage to elements of the exosuit 2900. For example, the hardware interface electronics 2940 can include a metal oxide varistor, breaker, shunt diode, or other element configured to limit the voltage and/or current applied to a winding of a motor.

Note that the above functions described as being enabled by the operating system 2912 can additionally or alternatively be implemented by applications 2914 a, 2914 b, 2914 c, services, drivers, daemons, or other computer-readable programs executed by the controller 2900. The applications, drivers, services, daemons, or other computer-readable programs can have special security privileges or other properties to facilitate their use to enable the above functions.

The operating system 2912 can encapsulate the functions of the hardware interface electronics 2940, actuators 2920, and sensors 2930 for use by other computer-readable programs (e.g., applications 2914 a, 2914 b, 2914 c, calibration service 2916), by the user (through the user interface 2950), and/or by some other system (i.e., a system configured to communicate with the controller 2910 through the communications interface 2960). The encapsulation of functions of the exosuit 2900 can take the form of application programming interfaces (APIs), i.e., sets of function calls and procedures that an application running on the controller 2910 can use to access the functionality of elements of the exosuit 2900. In some examples, the operating system 2912 can make available a standard ‘exosuit API’ to applications being executed by the controller 2910. The ‘exosuit API’ can enable applications 2914 a, 2914 b, 2914 c to access functions of the exosuit 2900 without requiring those applications 2914 a, 2914 b, 2914 c to be configured to generate whatever complex, time-dependent signals are necessary to operate elements of the exosuit 2900 (e.g., actuators 2920, sensors 2930).

The ‘exosuit API’ can allow applications 2914 a, 2914 b, 2914 c to send simple commands to the operating system 2912 (e.g., ‘begin storing mechanical energy from the ankle of the wearer when the foot of the wearer contacts the ground’) in such that the operating system 2912 can interpret those commands and generate the command signals to the hardware interface electronics 2940 or other elements of the exosuit 2900 that are sufficient to effect the simple commands generated by the applications 2914 a, 2914 b, 2914 c (e.g., determining whether the foot of the wearer has contacted the ground based on information detected by the sensors 2930, responsively applying high voltage to an exotendon that crosses the user's ankle).

The ‘exosuit API’ can be an industry standard (e.g., an ISO standard), a proprietary standard, an open-source standard, or otherwise made available to individuals that can then produce applications for exosuits. The ‘exosuit API’ can allow applications, drivers, services, daemons, or other computer-readable programs to be created that are able to operate a variety of different types and configurations of exosuits by being configured to interface with the standard ‘exosuit API’ that is implemented by the variety of different types and configurations of exosuits. Additionally or alternatively, the ‘exosuit API’ can provide a standard encapsulation of individual exosuit-specific actuators (i.e., actuators that apply forces to specific body segments, where differently-configured exosuits may not include an actuator that applies forces to the same specific body segments) and can provide a standard interface for accessing information on the configuration of whatever exosuit is providing the ‘exosuit API’. An application or other program that accesses the ‘exosuit API’ can access data about the configuration of the exosuit (e.g., locations and forces between body segments generated by actuators, specifications of actuators, locations and specifications of sensors) and can generate simple commands for individual actuators (e.g., generate a force of 30 newtons for 50 milliseconds) based on a model of the exosuit generated by the application and based on the information on the accessed data about the configuration of the exosuit. Additional or alternate functionality can be encapsulated by an ‘exosuit API’ according to an application.

Applications 2914 a, 2914 b, 2914 c can individually enable all or parts of the functions and operating modes of a flexible exosuit described herein. For example, an application can enable haptic control of a robotic system by transmitting postures, forces, torques, and other information about the activity of a wearer of the exosuit 2900 and by translating received forces and torques from the robotic system into haptic feedback applied to the wearer (i.e., forces and torques applied to the body of the wearer by actuators 2920 and/or haptic feedback elements). In another example, an application can enable a wearer to locomote more efficiently by submitting commands to and receiving data from the operating system 2912 (e.g., through an API) such that actuators 2920 of the exosuit 2900 assist the movement of the user, extract negative work from phases of the wearer's locomotion and inject the stored work to other phases of the wearer's locomotion, or other methods of operating the exosuit 2900. Applications can be installed on the controller 2910 and/or on a computer-readable storage medium included in the exosuit 2900 by a variety of methods. Applications can be installed from a removable computer-readable storage medium or from a system in communication with the controller 2910 through the communications interface 2960. In some examples, the applications can be installed from a web site, a repository of compiled or un-compiled programs on the Internet, an online store (e.g., Google Play, iTunes App Store), or some other source. Further, functions of the applications can be contingent upon the controller 2910 being in continuous or periodic communication with a remote system (e.g., to receive updates, authenticate the application, to provide information about current environmental conditions).

The exosuit 2900 illustrated in FIG. 29 is intended as an illustrative example. Other configurations of flexible exosuits and of operating systems, kernels, applications, drivers, services, daemons, or other computer-readable programs are anticipated. For example, an operating system configured to operate an exosuit can include a real-time operating system component configured to generate low-level commands to operate elements of the exosuit and a non-real-time component to enable less time-sensitive functions, like a clock on a user interface, updating computer-readable programs stored in the exosuit, or other functions. A exosuit can include more than one controller; further, some of those controllers can be configured to execute real-time applications, operating systems, drivers, or other computer-readable programs (e.g., those controllers were configured to have very short interrupt servicing routines, very fast thread switching, or other properties and functions relating to latency-sensitive computations) while other controllers are configured to enable less time-sensitive functions of a flexible exosuit. Additional configurations and operating modes of an exosuit are anticipated. Further, control systems configured as described herein can additionally or alternatively be configured to enable the operation of devices and systems other than exosuit; for example, control systems as described herein can be configured to operate robots, rigid exosuits or exoskeletons, assistive devices, prosthetics, or other mechatronic devices.

Control of actuators of an exosuit can be implemented in a variety of ways according to a variety of control schemes. Generally, one or more hardware and/or software controllers can receive information about the state of the flexible exosuit, a wearer of the exosuit, and/or the environment of the exosuit from sensors disposed on or within the exosuit and/or a remote system in communication with the exosuit. The one or more hardware and/or software controllers can then generate a control output that can be executed by actuators of the exosuit to affect a commanded state of the exosuit and/or to enable some other application. One or more software controllers can be implemented as part of an operating system, kernel, driver, application, service, daemon, or other computer-readable program executed by a processor included in the exosuit.

In some embodiments, a powered assistive exosuit intended primarily for assistive functions can also be adapted to perform exosuit functions. In one embodiment, an assistive exosuit similar to the embodiments described in U.S. Patent Application Publication No. 2018/0056104, titled “Systems and Methods for Assistive Exosuit System,” that is used for assistive functions may be adapted to perform exosuit functions. Embodiments of such an assistive exosuit typically include FLAs approximating muscle groups such as hip flexors, gluteal/hip extensors, spinal extensors, or abdominal muscles. In the assistive modes of these exosuits, these FLAs provide assistance for activities such as moving between standing and seated positions, walking, and postural stability. Actuation of specific FLAs within such an exosuit system may also provide stretching assistance. Typically, activation of one or more FLAs approximating a muscle group can stretch the antagonist muscles. For example, activation of one or more FLAs approximating the abdominal muscles might stretch the spinal extensors, or activation of one or more FLAs approximating gluteal/hip extensor muscles can stretch the hip flexors. The exosuit may be adapted to detect when the wearer is ready to initiate a stretch and perform an automated stretching regimen; or the wearer may indicate to the suit to initiate a stretching regimen.

It can be appreciated that assistive exosuits may have multiple applications. Assistive exosuits may be prescribed for medical applications. These may include therapeutic applications, such as assistance with exercise or stretching regimens for rehabilitation, disease mitigation or other therapeutic purposes. Mobility-assistance devices such as wheelchairs, walkers, crutches and scooters are often prescribed for individuals with mobility impairments. Likewise, an assistive exosuit may be prescribed for mobility assistance for patients with mobility impairments. Compared with mobility assistance devices such as wheelchairs, walkers, crutches and scooters, an assistive exosuit may be less bulky, more visually appealing, and conform with activities of daily living such as riding in vehicles, attending community or social functions, using the toilet, and common household activities.

An assistive exosuit may additionally function as primary apparel, fashion items or accessories. The exosuit may be stylized for desired visual appearance. The stylized design may reinforce visual perception of the assistance that the exosuit is intended to provide. For example, an assistive exosuit intended to assist with torso and upper body activities may present a visual appearance of a muscular torso and upper body. Alternatively, the stylized design may be intended to mask or camouflage the functionality of the assistive exosuit through design of the base layer, electro/mechanical integration or other design factors.

Similarly to assistive exosuits intended for medically prescribed mobility assistance, assistive exosuits may be developed and utilized for non-medical mobility assistance, performance enhancement and support. For many, independent aging is associated with greater quality of life, however activities may become more limited with time due to normal aging processes. An assistive exosuit may enable aging individuals living independently to electively enhance their abilities and activities. For example, gait or walking assistance could enable individuals to maintain routines such as social walking or golf. Postural assistance may render social situations more comfortable, with less fatigue. Assistance with transitioning between seated and standing positions may reduce fatigue, increase confidence, and reduce the risk of falls. These types of assistance, while not explicitly medical in nature, may enable more fulfilling, independent living during aging processes.

Athletic applications for an assistive exosuit are also envisioned. In one example, an exosuit may be optimized to assist with a particular activity, such as cycling. In the cycling example, FLAs approximating gluteal or hip extensor muscles may be integrated into bicycle clothing, providing assistance with pedaling. The assistance could be varied based on terrain, fatigue level or strength of the wearer, or other factors. The assistance provided may enable increased performance, injury avoidance, or maintenance of performance in the case of injury or aging. It can be appreciated that assistive exosuits could be optimized to assist with the demands of other sports such as running, jumping, swimming, skiing, or other activities. An athletic assistive exosuit may also be optimized for training in a particular sport or activity. Assistive exosuits may guide the wearer in proper form or technique, such as a golf swing, running stride, skiing form, swimming stroke, or other components of sports or activities. Assistive exosuits may also provide resistance for strength or endurance training. The provided resistance may be according to a regimen, such as high intensity intervals.

Assistive exosuit systems as described above may also be used in gaming applications. Motions of the wearer, detected by the suit, may be incorporated as a game controller system. For example, the suit may sense wearer's motions that simulate running, jumping, throwing, dancing, fighting, or other motions appropriate to a particular game. The suit may provide haptic feedback to the wearer, including resistance or assistance with the motions performed or other haptic feedback to the wearer.

Assistive exosuits as described above may be used for military or first responder applications. Military and first responder personnel are often to be required to perform arduous work where safety or even life may be at stake. An assistive exosuit may provide additional strength or endurance as required for these occupations. An assistive exosuit may connect to one or more communication networks to provide communication services for the wearer, as well as remote monitoring of the suit or wearer.

Assistive exosuits as described above may be used for industrial or occupational safety applications. Exosuits may provide more strength or endurance for specific physical tasks such as lifting or carrying or repetitive tasks such as assembly line work. By providing physical assistance, assistive exosuits may also help avoid or prevent occupational injury due overexertion or repetitive stress.

Assistive exosuits as described above may also be configured as home accessories. Home accessory assistive exosuits may assist with household tasks such as cleaning or yard work, or may be used for recreational or exercise purposes. The communication capabilities of an assistive exosuit may connect to a home network for communication, entertainment or safety monitoring purposes.

It is to be understood that the disclosed subject matter is not limited in its application to the details of construction and to the arrangements of the components set forth in the following description or illustrated in the drawings. The disclosed subject matter is capable of other embodiments and of being practiced and carried out in various ways. Also, it is to be understood that the phraseology and terminology employed herein are for the purpose of description and should not be regarded as limiting.

As such, those skilled in the art can appreciate that the conception, upon which this disclosure is based, may readily be utilized as a basis for the designing of other structures, systems, methods and media for carrying out the several purposes of the disclosed subject matter.

Although the disclosed subject matter has been described and illustrated in the foregoing exemplary embodiments, it is understood that the present disclosure has been made only by way of example, and that numerous changes in the details of implementation of the disclosed subject matter may be made without departing from the spirit and scope of the disclosed subject matter.

What is claimed is:

1. A device for use with an exosuit comprising:
a housing;
communications circuitry operative to communicate with the exosuit;
an interactive display exposed through an opening of the housing; and
a processor positioned within the housing and is configured to:
display, on the interactive display, a plurality of exosuit control buttons, wherein each exosuit control button corresponds to at least one power layer of the exosuit responsible for implementing exosuit assistance associated with that particular exosuit control button;
receive data from the exosuit via the communications circuitry, the data comprising data corresponding to the at least one power layer associated with each of the exosuit control buttons; and
change a visual element of at least one of the exosuit control buttons based on the received data.

2. The device of claim 1, wherein the visual element comprises a countdown timer that graphically illustrates when the at least one power layer associated with one of exosuit control buttons is going to activate.

3. The device of claim 2, wherein when the countdown timer expires, the at least one power layer associated with one of the exosuit control buttons is activated.

4. The device of claim 1, wherein the visual element changes from an inactive state to an active state, wherein in the inactive state, the at least one power layer associated with one of the exosuit control buttons is not activated, and wherein in the active state, the at least one power layer associated with one of the exosuit control buttons is activated.

5. The device of claim 1, wherein the processor is further configured to:
receive user selection of one of the displayed plurality of exosuit control buttons; and
display, on the interactive display, a configuration screen comprising:
an ON/OFF toggle switch operative to enable or disable the at least one power layer associated with the selected exosuit control button; and
a force quantity control to set a level of assistance force applied by the at least one power layer associated with the selected exosuit control button.

6. The device of claim 1, wherein the processor is further configured to:
receive a user swipe input in a first direction; and
display, on the interactive display, battery status of at least one of the power layers in response to the user swipe input in the first direction, wherein the battery status of each of the power layers is received from the exosuit via the communications circuitry.

7. The device of claim 1, wherein the processor is further configured to:
receive a user swipe input in a second direction; and
display, on the interactive display, a settings screen comprising:
a posture calibration button that, when selected, is operative to calibrate the exosuit; and
an exosuit pairing button that, when selected, is operative to enable a user to pair the device with the exosuit.

8. The device of claim 1, wherein the processor is further configured to:
display, on the interactive display, a symbiosis button;
receive user selection of the symbiosis button;
display, on the interactive display, in response to user selection of the symbiosis button a scrollable list comprising:
a plurality of exosuit assistance movement buttons each associated with an ON/OFF toggle switch; and
a plurality of timer buttons corresponding to respective ones of the plurality of exosuit control buttons, wherein each one of the plurality of timer buttons specifies a time limit and is further selectable to enable a user to define the time limit.

9. A method for operating an accessory device that is in communication with an exosuit system, the method comprising:
initiating a symbiosis mode of control for the exosuit in response to user selection of a symbiosis button on the accessory device, the symbiosis mode of control comprising:
receiving data from the exosuit system, the data comprising a determined body posture of the user wearing the exosuit suit;
displaying, on an interactive display of the accessory device, a first exosuit control button comprising a first timer;
activating the first timer in response to receipt of the determined body posture, wherein activating the first timer comprises displaying a countdown of the first timer and during the countdown of the first timer, the exosuit system is configuring at least one flexible linear actuator associated with at least one power layer to actuate a base tone commensurate with the determined body posture; and
when the first timer has elapsed, changing a display element of the first exosuit control button to indicate that exosuit enabled assistance support is ready for activation, and wherein when exosuit enabled assistance support is triggered to go active, the exosuit system transitions from the base tone to active assistance support.

10. The method of claim 9, the symbiosis mode of control further comprising:
displaying, on the interactive display, a cancel button;
receiving user selection of the cancel button, wherein selection of the cancel button ends the active assistance support; and
changing the display element to indicate the exosuit enabled assistance support is not active in response to user selection of the cancel button.

11. The method of claim 9, the symbiosis mode of control further comprising:
receiving a new determined body posture while the first timer is counting down;
resetting the first timer in response to receipt of the new determined body posture; and
re-activating the first timer after the first timer has been reset and displaying the countdown of the first timer.

12. The method of claim 9, wherein the determined body posture is a physiological determination of a positional state or predicted motion state of a user of the exosuit system.

13. The method of claim 9, wherein the base tone comprises pre-emptive tensioning of the at least one flexible linear actuator that is used to provide the exosuit enabled assistance support.

14. The method of claim 9, wherein a time duration of the first timer is an amount of time configurable by a user via manipulation of a setting in the interactive display.

15. The method of claim 14, wherein the time duration is the amount of time the user remained in the determined posture in order to transition to the base tone.

16. The method of claim 15, the symbiosis mode of control further comprising:
receiving a trigger to transition from the base tone to the active assistance support; and
operating the at least one power layer to provide the active assistance support.

17. The method of claim 16, wherein the trigger is an elapse of the first timer.

18. The method of claim 16, wherein the trigger is included in the received data provided by the exosuit system or is a input received via the interactive display.

19. A method for operating an accessory device that is in communication with an exosuit system, the method comprising:
displaying, on an interactive display, a home page comprising a symbiosis/manual toggle switch and a plurality of exosuit control buttons, wherein each of the exosuit control buttons corresponds to an exosuit assistance operation;
receiving user selection of the symbiosis/manual toggle switch to operate the exosuit system in a manual mode, wherein the manual mode requires the user to select one of the exosuit control buttons to activate the exosuit assistance operation corresponding to the selected exosuit control button; and
receiving user selection of the symbiosis/manual toggle switch to operate the exosuit system in a symbiosis mode, wherein the symbiosis mode automatically activates an exosuit assistance operation based on data received from the exosuit system, and
wherein the exosuit control button corresponding to the exosuit assistance operation is displayed with at least one display element that changes depending on a status of the exosuit control button.

20. The method of claim 19, wherein the at least one display element comprises a countdown timer that indicates when the exosuit assistance operation is ready for activation, the method further comprising displaying a configuration screen corresponding to a selected one of the plurality of exosuit control buttons, the configuration screen comprising a force quantity control to set a level of assistance force applied by the exosuit suit during activation of the exosuit assistance operation corresponding to the selected exosuit control button.
